(19) United States
(12) Patent Application Publication
(10) Pub. No.: US 2018/0240140 A1
(43) Pub. Date: Aug. 23, 2018

(54) Systems and Methods for Analytics and Gamification of Healthcare

(71) Applicant: General Electric Company, Schenectady, NY (US)

(72) Inventors: Garry Whitley, Clinton, TN (US); Travis Frosch, Orlando, FL (US); Christopher Holly, San Ramon, CA (US)

(21) Appl. No.: 15/594,335

(22) Filed: May 12, 2017

Related U.S. Application Data
(60) Provisional application No. 62/460,595, filed on Feb. 17, 2017.

(51) Int. Cl. G06Q 30/02; G06F 19/00; A63F 13/80; A63F 13/46; G09B 19/00

(52) U.S. Cl. CPC G06Q 30/0209; G06F 19/327; A63F 13/80; G06Q 10/063114; G09B 19/00; G06F 19/321; A63F 13/46

(57) ABSTRACT

Certain examples provide systems and methods for analytics and gamification of healthcare workflows. Certain examples provide an apparatus including a game engine to at least: process a healthcare workflow to form a processed workflow organized according to task, each task electronically represented in the processed workflow; apply game rules to the processed workflow to form a gamified workflow, the game rules associating a value with each task in the processed workflow, the value electronically represented with the corresponding task in the gamified workflow; and define a goal and a reward based on the gamified workflow. The example apparatus also includes a process tracker to at least: track progress with respect to the goal during execution of the gamified workflow; generate a score based on the monitored progress of the execution of the gamified workflow; and provide feedback and reward based on a comparison of the score to the goal.

### CROSS-REFERENCE TO RELATED APPLICATIONS

This patent arises from U.S. Provisional Patent Application No. 62/460,595, entitled “SYSTEMS AND METHODS FOR ANALYTICS AND GAMIFICATION OF HEALTHCARE”, filed Feb. 17, 2017, which is incorporated herein by reference in its entirety.

### BACKGROUND

Many millennials are entering the job force for the first time, and the associated data is staggering. Millennials—those aged 18 to 34—represent 2.4 billion of the world's population. Millennials number greater than 83 million in the U.S. alone. In just nine years, they will make up more than 75% of the global workforce.

A recent Gallup report estimated that 71% of millennial workers are not fully engaged at work. When millennials are not fully engaged, they do not remain with that employer. Studies have indicated that more than 60% of millennials leave their employers within 3 years. The replacement cost for their ex-employers ranges from $15,000 to $25,000 per lost employee.

In a challenging field, such as healthcare, a methodology and technology to engage their profession workforce is lacking and unavailable and/or insufficient to incentivize better use of healthcare systems and associated workflows.

### BRIEF DESCRIPTION

Certain examples provide systems and methods for analytics and gamification of healthcare workflows.

Certain examples provide an apparatus including a game engine including a processor configured to at least: process a healthcare workflow to form a processed workflow organized according to task, each task electronically represented in the processed workflow; apply game rules to the processed workflow to form a gamified workflow, the game rules associating a value with each task in the processed workflow, the value electronically represented with the corresponding task in the gamified workflow; and define a goal and a reward based on the gamified workflow. The example apparatus also includes a process tracker including a processor configured to at least: track progress with respect to the goal during execution of the gamified workflow including each task; generate a score based on the monitored progress of the execution of the gamified workflow; and provide feedback and reward based on a comparison of the score to the goal.

Certain examples provide a method. The example method includes processing, using a game engine including a processor, a healthcare workflow to form a processed workflow organized according to task, each task electronically represented in the processed workflow. The example method includes applying, using the processor, game rules to the processed workflow to form a gamified workflow, the game rules associating a value with each task in the processed workflow, the value electronically represented with the corresponding task in the gamified workflow. The example method includes defining, using the processor, a goal and a reward based on the gamified workflow. The example method includes tracking, using the processor, progress with respect to the goal during execution of the gamified workflow including each task. The example method includes generating, using the processor, a score based on the monitored progress of the execution of the gamified workflow. The example method includes providing, using the processor, feedback and reward based on a comparison of the score to the goal.

Certain examples provide a computer-readable storage medium including instructions which, when executed, configure a processor to at least implement a system. The example system includes a game engine configured to at least: process a healthcare workflow to form a processed workflow organized according to task, each task electronically represented in the processed workflow; apply game rules to the processed workflow to form a gamified workflow, the game rules associating a value with each task in the processed workflow, the value electronically represented with the corresponding task in the gamified workflow; and define a goal and a reward based on the gamified workflow. The example system also includes a process tracker configured to at least: track progress with respect to the goal during execution of the gamified workflow including each task; generate a score based on the monitored progress of the execution of the gamified workflow; and provide feedback and reward based on a comparison of the score to the goal.

### BRIEF DESCRIPTION OF THE DRAWINGS

The features and technical aspects of the system and method disclosed herein will become apparent in the following Detailed Description set forth below when taken in conjunction with the drawings in which like reference numerals indicate identical or functionally similar elements.

FIG. 1 shows a block diagram of an example healthcare-focused information system.

FIG. 2 shows a block diagram of an example healthcare information infrastructure.

FIG. 3 illustrates an example industrial internet configuration.

FIG. 4 illustrates an example healthcare gamification system.

FIG. 5 illustrates an example implementation of the organizational review and goal setting module of FIG. 4.

FIG. 6 illustrates an example implementation of the game engine of FIG. 5.

FIG. 7 illustrates an example implementation of the process tracking engine of FIG. 4.

FIG. 8 illustrates an example implementation of the example reward and behavior modification engine of FIG. 4.

FIG. 9 illustrates an example group view leaderboard tracker.

FIG. 10 illustrates a particular example implementation of the tracker of FIG. 7

FIG. 11 illustrates an example end user dashboard.

FIG. 12 illustrates an example user board.

FIG. 13 illustrates an example weekly objectives dashboard.

FIG. 14 illustrates an example cash by game interface.

FIG. 15 provides a closer view of an example dashboard.

FIG. 16 shows an example achievement feed for a dashboard.

FIGS. 17-19 illustrate flow diagrams of example methods to provide analytics and gamification in a healthcare workflow.

FIGS. 20-21 are block diagrams of example processor platforms capable of executing instructions to implement the example systems and methods of FIGS. 1-19.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

### I. Overview

Gamification

Gamification is an application of elements of game playing (e.g., point scoring, competition with others, rules of play, etc.) to other areas of activity. Certain examples provide gamification to healthcare workflows using analytics to drive technological functional improvements and display changes for such workflows.

Certain examples enhance employee engagement with healthcare delivery technologies and processes. Certain examples solve the vexing problem of engaging millennials (e.g., people aged 18-34, representing 2.4 billion people on the planet) in healthcare jobs through technological improvements to computing infrastructure and graphical user interface (GUI) program interaction.

Certain examples provide improved analytics in a scalable, integrated workflow solution. Analytics dashboards can be generated to provide progress and leaderboard information including badge systems and visible indicators regarding progress toward objectives, job responsibilities, clinical protocol tasks, etc., as embedded directly into a workflow application.

Certain examples provide a user interface (e.g., including a set of dashboards for specific use-cases and/or personas, etc.), a back-end data structure to power the solution and store related data, and an interface for client administrators to update scores and/or rewards relative to specific activities.

Organizations are facing many significant challenges arising in healthcare as costs to provide high quality healthcare are higher than ever. A first problem facing healthcare organizations is an inability to identify bottlenecks or blackholes in healthcare processes and/or workflows. Certain examples address this problem through improved workflow visualization, progress tracking, and analytics. A second problem facing healthcare organization is keeping an ever-increasing number of millennial employees engaged with their work to accomplish daily tasks. Certain examples address this problem by providing new dashboards, analytics, and improved workflows to facilitate and maintain user engagement.

Certain examples facilitate identification of near real-time status (e.g., 1-minute data latency, 5-minute data latency, 10-minute data latency, etc.) including: a) where tasks currently reside in a healthcare workflow, b) an owner of each task, and c) a total dollar amount in dollars related to each stratification of the data. In certain examples, administrative dashboards allow supervisors to determine, without escalation, where workflows are dead-ending and/or where dollars are piling up. Supervisors can then act with their teams and/or individuals to impact a desired employee behavior. In certain examples, a reward table can be manipulated within the application to positively impact the desired employee behavior. For example, a reward value for a given task that may make a positive impact can be raised, and/or a reward value for a task that brings a negative impact can be reduced.

Certain examples provide dashboards to keep employees more engaged with their work and driving to meet goals set by managerial team(s). For example, each individual staff member (e.g., healthcare gamer) has a dashboard which is specific to his or her performance. The dashboard displays an avatar which has been selected by the user (e.g., the staff member), the user's name, specific management game bonus(es), a display of badge(s) that the user may have recently earned and some additional metric(s), for example. Users can see where they rank against their peers and where they stand in relation to their weekly or monthly objective (e.g., determined by management teams). Two other widgets (e.g., sections of the dashboard) that drive competitive nature are a) an individual leaderboard, which shows the top gamers with overall points scored and b) an activity ticker showing gamers that have reached a specific achievement goal in reverse chronological order (e.g., most current at the top of the feed, etc.).

For example, a user board provides scoring and status information as well as goals (e.g., organizational goals, team goals, individual goals, etc.). The example user board can generate, track, and display management and/or organizational goals such as paid in full, desired final outcome, etc. The example user board can generate, track, and display individual and/or team goals such as fastest person to close 100 tasks this week, above and beyond awards, etc.

A second dashboard is a display created for common areas, such a break rooms or combined office settings. This dashboard displays the gamers currently meeting or exceeding a current challenge, the activity feed, and other metrics around overall activity of the business(es).

In operation, data is (repeatedly) extracted from one or more healthcare workflow software applications. These extracts are then moved into a centralized location where they can then be loaded into an optimized analytics data structure. The dashboards are connected into the analytics data structures to provide visualizations of the data to end users.

In certain examples, a reward table is used as a meta-data table to derive points or coins incremented or decremented against a number of records in each user's task activity. Such accounting with respect to the records allows a management team to change the meta data, which impacts the scoring system. The data can be manipulated to drive desired behavior characteristics of the staff. Visualizations are delivered over a secure web address (e.g., uniform resource locator (URL), etc.) and allow for filtering, drilling into detail data (if enabled) and exporting of the widgets or the whole dashboard into an image file, portable document format (PDF) file, or the underlying data in comma separated values (.csv format), etc.

Using the reward table, leader/user board, database information, and healthcare workflow information, certain examples facilitate activity-based costing. That is, certain examples generate an alphanumeric quantification and visual qualification of how much a portion (e.g., a slice) of a healthcare process/workflow costs to deliver, and helps an organization track that activity versus cost. For each healthcare user, buckets, slices, or queues can be formed and displayed to allow insight into where and how much money is getting stuck in queues for a particular user or group of users, for example.

In certain examples, pie charts and/or other tables are generated and displayed with an ability to drill down to determine a last task a user was working on and the task's status (e.g., completed, in progress, on hold, etc.). In certain examples, associated cash tied up awaiting completion of the task can be viewed and analyzed, along with its progress or pendency (e.g., in progress for 60 days, 90 days, 120 days, etc.). In certain examples, a penalty can be assessed if a deadline/due date is moved, if a user falls behind or is late, etc. Thus, certain examples supplement and/or otherwise extend business intelligence software (e.g., Sisense, etc.) to provide technological improvements in healthcare workflow tracking, processing, analytics, and user motivation/development.

Certain examples process available healthcare workflow data in a time direction. For example, task status and associated badges earned are determined to distribute an achievement list over time. A task can be checked against when a badge was earned over time, for example.

In certain examples, healthcare users can be represented by avatars displayed via the dashboard interface(s). The avatars can represent the users and reflect changing user roles, priorities, status, etc.

As will be described further below, certain examples can integrate with and operate in a variety of healthcare environments and impact a variety of healthcare scenarios and data through sensing, decision support, workflow management, and control. The following section provides some context and example environment for the presently disclosed technology described further in the subsequent section below.

### II. Example Operating Environments

Health information, also referred to as healthcare information and/or healthcare data, relates to information generated and/or used by a healthcare entity. Health information can be information associated with health of one or more patients, for example. Health information may include protected health information (PHI), as outlined in the Health Insurance Portability and Accountability Act (HIPAA), which is identifiable as associated with a particular patient and is protected from unauthorized disclosure. Health information can be organized as internal information and external information. Internal information includes patient encounter information (e.g., patient-specific data, aggregate data, comparative data, etc.) and general healthcare operations information, etc. External information includes comparative data, expert and/or knowledge-based data, etc. Information can have both a clinical (e.g., diagnosis, treatment, prevention, etc.) and administrative (e.g., scheduling, billing, management, etc.) purpose.

Institutions, such as healthcare institutions, having complex network support environments and sometimes chaotically driven process flows utilize secure handling and safeguarding of the flow of sensitive information (e.g., personal privacy). A need for secure handling and safeguarding of information increases as a demand for flexibility, volume, and speed of exchange of such information grows. For example, healthcare institutions provide enhanced control and safeguarding of the exchange and storage of sensitive patient PHI and employee information between diverse locations to improve hospital operational efficiency in an operational environment typically having a chaotic-driven demand by patients for hospital services. In certain examples, patient identifying information can be masked or even stripped from certain data depending upon where the data is stored and who has access to that data. In some examples, PHI that has been “de-identified” can be re-identified based on a key and/or other encoder/decoder.

A healthcare information technology infrastructure can be adapted to service multiple business interests while providing clinical information and services. Such an infrastructure may include a centralized capability including, for example, a data repository, reporting, discrete data exchange/connectivity, “smart” algorithms, personalization/consumer decision support, etc. This centralized capability provides information and functionality to a plurality of users including medical devices, electronic records, access portals, pay for performance (P4P), chronic disease models, and clinical health information exchange/regional health information organization (HIE/RHIO), and/or enterprise pharmaceutical studies, home health, for example.

Interconnection of multiple data sources helps enable an engagement of all relevant members of a patient's care team and helps improve an administrative and management burden on the patient for managing his or her care. Particularly, interconnecting the patient's electronic medical record and/or other medical data can help improve patient care and management of patient information. Furthermore, patient care compliance is facilitated by providing tools that automatically adapt to the specific and changing health conditions of the patient and provide comprehensive education and compliance tools to drive positive health outcomes.

In certain examples, healthcare information can be distributed among multiple applications using a variety of database and storage technologies and data formats. To provide a common interface and access to data residing across these applications, a connectivity framework (CF) can be provided which leverages common data and service models (CDM and CSM) and service oriented technologies, such as an enterprise service bus (ESB) to provide access to the data.

In certain examples, a variety of user interface frameworks and technologies can be used to build applications for health information systems including, but not limited to, MICROSOFT® ASP.NET, AJAX®, MICROSOFT® Windows Presentation Foundation, GOOGLE® Web Toolkit, MICROSOFT® Silverlight, ADOBE®, and others. Applications can be composed from libraries of information widgets to display multi-content and multi-media information, for example. In addition, the framework enables users to tailor layout of applications and interact with underlying data.

In certain examples, an advanced Service-Oriented Architecture (SOA) with a modern technology stack helps provide robust interoperability, reliability, and performance. Example SOA includes a three-fold interoperability strategy including a central repository (e.g., a central repository built from Health Level Seven (HL7) transactions), services for working in federated environments, and visual integration with third-party applications. Certain examples provide portable content enabling plug 'n play content exchange among healthcare organizations. A standardized vocabulary using common standards (e.g., LOINC, SNOMED CT, RxNorm, FDB, ICD-9, ICD-10, etc.) is used for interoperability, for example. Certain examples provide an intuitive user interface to help minimize end-user training. Certain examples facilitate user-initiated launching of third-party applications directly from a desktop interface to help provide a seamless workflow by sharing user, patient, and/or other contexts. Certain examples provide real-time (or at least substantially real time assuming some system delay) patient data from one or more information technology (IT) systems and facilitate comparison(s) against evidence-based best practices. Certain examples provide one or more dashboards for specific sets of patients. Dashboard(s) can be based on condition, role, and/or other criteria to indicate variation(s) from a desired practice, for example.

### A. Example Healthcare Information System

An information system can be defined as an arrangement of information/data, processes, and information technology that interact to collect, process, store, and provide informational output to support delivery of healthcare to one or more patients. Information technology includes computer technology (e.g., hardware and software) along with data and telecommunications technology (e.g., data, image, and/or voice network, etc.).

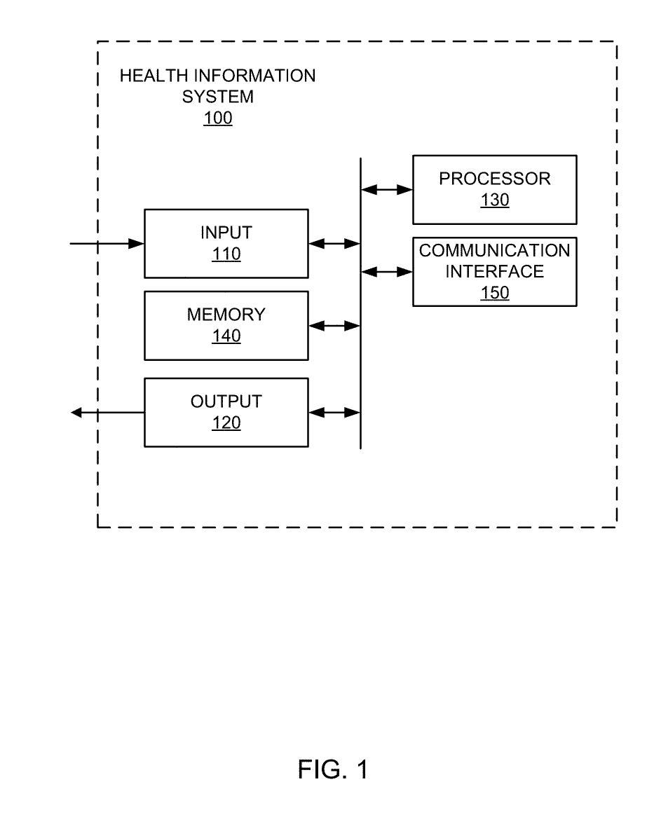

Turning now to the figures, FIG. 1 shows a block diagram of an example healthcare-focused information system 100. Example system 100 can be configured to implement a variety of systems and processes including image storage (e.g., picture archiving and communication system (PACS), etc.), image processing and/or analysis, radiology reporting and/or review (e.g., radiology information system (RIS), etc.), computerized provider order entry (CPOE) system, clinical decision support, patient monitoring, population health management (e.g., population health management system (PHMS), health information exchange (HIE), etc.), healthcare data analytics, cloud-based image sharing, electronic medical record (e.g., electronic medical record system (EMR), electronic health record system (EHR), electronic patient record (EPR), personal health record system (PHR), etc.), and/or other health information system (e.g., clinical information system (CIS), hospital information system (HIS), patient data management system (PDMS), laboratory information system (LIS), cardiovascular information system (CVIS), etc.

As illustrated in FIG. 1, the example information system 100 includes an input 110, an output 120, a processor 130, a memory 140, and a communication interface 150. The components of example system 100 can be integrated in one device or distributed over two or more devices.

Example input 110 may include a keyboard, a touch-screen, a mouse, a trackball, a track pad, optical barcode recognition, voice command, etc. or combination thereof used to communicate an instruction or data to system 100. Example input 110 may include an interface between systems, between user(s) and system 100, etc.

Example output 120 can provide a display generated by processor 130 for visual illustration on a monitor or the like. The display can be in the form of a network interface or graphic user interface (GUI) to exchange data, instructions, or illustrations on a computing device via communication interface 150, for example. Example output 120 may include a monitor (e.g., liquid crystal display (LCD), plasma display, cathode ray tube (CRT), etc.), light emitting diodes (LEDs), a touch-screen, a printer, a speaker, or other conventional display device or combination thereof.

Example processor 130 includes hardware and/or software configuring the hardware to execute one or more tasks and/or implement a particular system configuration. Example processor 130 processes data received at input 110 and generates a result that can be provided to one or more of output 120, memory 140, and communication interface 150. For example, example processor 130 can take user annotation provided via input 110 with respect to an image displayed via output 120 and can generate a report associated with the image based on the annotation. As another example, processor 130 can process imaging protocol information obtained via input 110 to provide an updated configuration for an imaging scanner via communication interface 150.

Example memory 140 may include a relational database, an object-oriented database, a data dictionary, a clinical data repository, a data warehouse, a data mart, a vendor neutral archive, an enterprise archive, etc. Example memory 140 stores images, patient data, best practices, clinical knowledge, analytics, reports, etc. Example memory 140 can store data and/or instructions for access by the processor 130. In certain examples, memory 140 can be accessible by an external system via the communication interface 150.

Example communication interface 150 facilitates transmission of electronic data within and/or among one or more systems. Communication via communication interface 150 can be implemented using one or more protocols. In some examples, communication via communication interface 150 occurs according to one or more standards (e.g., Digital Imaging and Communications in Medicine (DICOM), Health Level Seven (HL7), ANSI X12N, etc.). Example communication interface 150 can be a wired interface (e.g., a data bus, a Universal Serial Bus (USB) connection, etc.) and/or a wireless interface (e.g., radio frequency, infrared (IR), near field communication (NFC), etc.). For example, communication interface 150 may communicate via wired local area network (LAN), wireless LAN, wide area network (WAN), etc. using any past, present, or future communication protocol (e.g., BLUETOOTH™, USB 2.0, USB 3.0, etc.).

In certain examples, a Web-based portal may be used to facilitate access to information, protocol library, imaging system configuration, patient care and/or practice management, etc. Information and/or functionality available via the Web-based portal may include one or more of order entry, laboratory test results review system, patient information, clinical decision support, medication management, scheduling, electronic mail and/or messaging, medical resources, etc. In certain examples, a browser-based interface can serve as a zero footprint, zero download, and/or other universal viewer for a client device.

In certain examples, the Web-based portal serves as a central interface to access information and applications, for example. Data may be viewed through the Web-based portal or viewer, for example. Additionally, data may be manipulated and propagated using the Web-based portal, for example. Data may be generated, modified, stored and/or used and then communicated to another application or system to be modified, stored and/or used, for example, via the Web-based portal, for example.

The Web-based portal may be accessible locally (e.g., in an office) and/or remotely (e.g., via the Internet and/or other private network or connection), for example. The Web-based portal may be configured to help or guide a user in accessing data and/or functions to facilitate patient care and practice management, for example. In certain examples, the Web-based portal may be configured according to certain rules, preferences and/or functions, for example. For example, a user may customize the Web portal according to particular desires, preferences and/or requirements.

### B. Example Healthcare Infrastructure

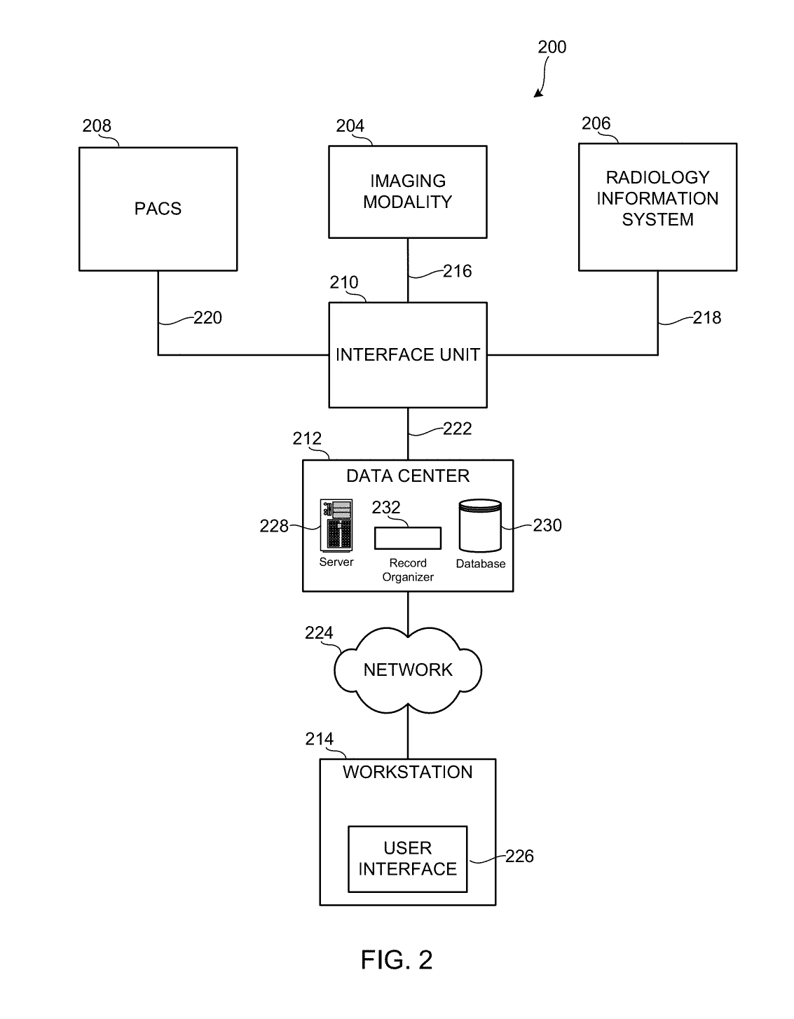

FIG. 2 shows a block diagram of an example healthcare information infrastructure 200 including one or more subsystems such as the example healthcare-related information system 100 illustrated in FIG. 1. Example healthcare system 200 includes an imaging modality 204, a RIS 206, a PACS 208, an interface unit 210, a data center 212, and a workstation 214. In the illustrated example, scanner/modality 204, RIS 206, and PACS 208 are housed in a healthcare facility and locally archived. However, in other implementations, imaging modality 204, RIS 206, and/or PACS 208 may be housed within one or more other suitable locations. In certain implementations, one or more of PACS 208, RIS 206, modality 204, etc., may be implemented remotely via a thin client and/or downloadable software solution. Furthermore, one or more components of the healthcare system 200 can be combined and/or implemented together. For example, RIS 206 and/or PACS 208 can be integrated with the imaging scanner 204; PACS 208 can be integrated with RIS 206; and/or the three example systems 204, 206, and/or 208 can be integrated together. In other example implementations, healthcare system 200 includes a subset of the illustrated systems 204, 206, and/or 208. For example, healthcare system 200 may include only one or two of the modality 204, RIS 206, and/or PACS 208. Information (e.g., scheduling, test results, exam image data, observations, diagnosis, etc.) can be entered into the scanner 204, RIS 206, and/or PACS 208 by healthcare practitioners (e.g., radiologists, physicians, and/or technicians) and/or administrators before and/or after patient examination. One or more of the imaging scanner 204, RIS 206, and/or PACS 208 can communicate with equipment and system(s) in an operating room, patient room, etc., to track activity, correlate information, generate reports and/or next actions, and the like.

The RIS 206 stores information such as, for example, radiology reports, radiology exam image data, messages, warnings, alerts, patient scheduling information, patient demographic data, patient tracking information, and/or physician and patient status monitors. Additionally, RIS 206 enables exam order entry (e.g., ordering an x-ray of a patient) and image and film tracking (e.g., tracking identities of one or more people that have checked out a film). In some examples, information in RIS 206 is formatted according to the HL-7 (Health Level Seven) clinical communication protocol. In certain examples, a medical exam distributor is located in RIS 206 to facilitate distribution of radiology exams to a radiologist workload for review and management of the exam distribution by, for example, an administrator.

PACS 208 stores medical images (e.g., x-rays, scans, three-dimensional renderings, etc.) as, for example, digital images in a database or registry. In some examples, the medical images are stored in PACS 208 using the Digital Imaging and Communications in Medicine (DICOM) format. Images are stored in PACS 208 by healthcare practitioners (e.g., imaging technicians, physicians, radiologists) after a medical imaging of a patient and/or are automatically transmitted from medical imaging devices to PACS 208 for storage. In some examples, PACS 208 can also include a display device and/or viewing workstation to enable a healthcare practitioner or provider to communicate with PACS 208.

The interface unit 210 includes a hospital information system interface connection 216, a radiology information system interface connection 218, a PACS interface connection 220, and a data center interface connection 222. Interface unit 210 facilities communication among imaging modality 204, RIS 206, PACS 208, and/or data center 212. Interface connections 216, 218, 220, and 222 can be implemented by, for example, a Wide Area Network (WAN) such as a private network or the Internet. Accordingly, interface unit 210 includes one or more communication components such as, for example, an Ethernet device, an asynchronous transfer mode (ATM) device, an 802.11 device, a DSL modem, a cable modem, a cellular modem, etc. In turn, the data center 212 communicates with workstation 214, via a network 224, implemented at a plurality of locations (e.g., a hospital, clinic, doctor's office, other medical office, or terminal, etc.). Network 224 is implemented by, for example, the Internet, an intranet, a private network, a wired or wireless Local Area Network, and/or a wired or wireless Wide Area Network. In some examples, interface unit 210 also includes a broker (e.g., a Mitra Imaging's PACS Broker) to allow medical information and medical images to be transmitted together and stored together.

Interface unit 210 receives images, medical reports, administrative information, exam workload distribution information, and/or other clinical information from the information systems 204, 206, 208 via the interface connections 216, 218, 220. If necessary (e.g., when different formats of the received information are incompatible), interface unit 210 translates or reformats (e.g., into Structured Query Language (“SQL”) or standard text) the medical information, such as medical reports, to be properly stored at data center 212. The reformatted medical information can be transmitted using a transmission protocol to enable different medical information to share common identification elements, such as a patient name or social security number. Next, interface unit 210 transmits the medical information to data center 212 via data center interface connection 222. Finally, medical information is stored in data center 212 in, for example, the DICOM format, which enables medical images and corresponding medical information to be transmitted and stored together.

The medical information is later viewable and easily retrievable at workstation 214 (e.g., by their common identification element, such as a patient name or record number). Workstation 214 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. Workstation 214 receives commands and/or other input from a user via, for example, a keyboard, mouse, track ball, microphone, etc. Workstation 214 is capable of implementing a user interface 226 to enable a healthcare practitioner and/or administrator to interact with healthcare system 200. For example, in response to a request from a physician, user interface 226 presents a patient medical history. In other examples, a radiologist is able to retrieve and manage a workload of exams distributed for review to the radiologist via user interface 226. In further examples, an administrator reviews radiologist workloads, exam allocation, and/or operational statistics associated with the distribution of exams via user interface 226. In some examples, the administrator adjusts one or more settings or outcomes via user interface 226.

Example data center 212 of FIG. 2 is an archive to store information such as images, data, medical reports, and/or, more generally, patient medical records. In addition, data center 212 can also serve as a central conduit to information located at other sources such as, for example, local archives, hospital information systems/radiology information systems (e.g., HIS 204 and/or RIS 206), or medical imaging/storage systems (e.g., PACS 208 and/or connected imaging modalities). That is, the data center 212 can store links or indicators (e.g., identification numbers, patient names, or record numbers) to information. In the illustrated example, data center 212 is managed by an application server provider (ASP) and is located in a centralized location that can be accessed by a plurality of systems and facilities (e.g., hospitals, clinics, doctor's offices, other medical offices, and/or terminals). In some examples, data center 212 can be spatially distant from the imaging modality 204, RIS 206, and/or PACS 208. In certain examples, the data center 212 can be located in the cloud (e.g., on a cloud-based server, an edge device, etc.).

Example data center 212 of FIG. 2 includes a server 228, a database 230, and a record organizer 232. Server 228 receives, processes, and conveys information to and from the components of healthcare system 200. Database 230 stores the medical information described herein and provides access thereto. Example record organizer 232 of FIG. 2 manages patient medical histories, for example. Record organizer 232 can also assist in procedure scheduling, for example.

Certain examples can be implemented as cloud-based clinical information systems and associated methods of use. An example cloud-based clinical information system enables healthcare entities (e.g., patients, clinicians, sites, groups, communities, and/or other entities) to share information via web-based applications, cloud storage and cloud services. For example, the cloud-based clinical information system may enable a first clinician to securely upload information into the cloud-based clinical information system to allow a second clinician to view and/or download the information via a web application. Thus, for example, the first clinician may upload an x-ray imaging protocol into the cloud-based clinical information system, and the second clinician may view and download the x-ray imaging protocol via a web browser and/or download the x-ray imaging protocol onto a local information system employed by the second clinician.

In certain examples, users (e.g., a patient and/or care provider) can access functionality provided by system 200 via a software-as-a-service (SaaS) implementation over a cloud or other computer network, for example. In certain examples, all or part of system 200 can also be provided via platform as a service (PaaS), infrastructure as a service (IaaS), etc. For example, system 200 can be implemented as a cloud-delivered Mobile Computing Integration Platform as a Service. A set of consumer-facing Web-based, mobile, and/or other applications enable users to interact with the PaaS, for example.

### C. Industrial Internet Examples

The Internet of things (also referred to as the “Industrial Internet”) relates to an interconnection between a device that can use an Internet connection to talk with other devices on the network. Using the connection, devices can communicate to trigger events/actions (e.g., changing temperature, turning on/off, providing a status, etc.). In certain examples, machines can be merged with “big data” to improve efficiency and operations, provide improved data mining, facilitate better operation, etc.

Big data can refer to a collection of data so large and complex that it becomes difficult to process using traditional data processing tools/methods. Challenges associated with a large data set include data capture, sorting, storage, search, transfer, analysis, and visualization. A trend toward larger data sets is due at least in part to additional information derivable from analysis of a single large set of data, rather than analysis of a plurality of separate, smaller data sets. By analyzing a single large data set, correlations can be found in the data, and data quality can be evaluated.

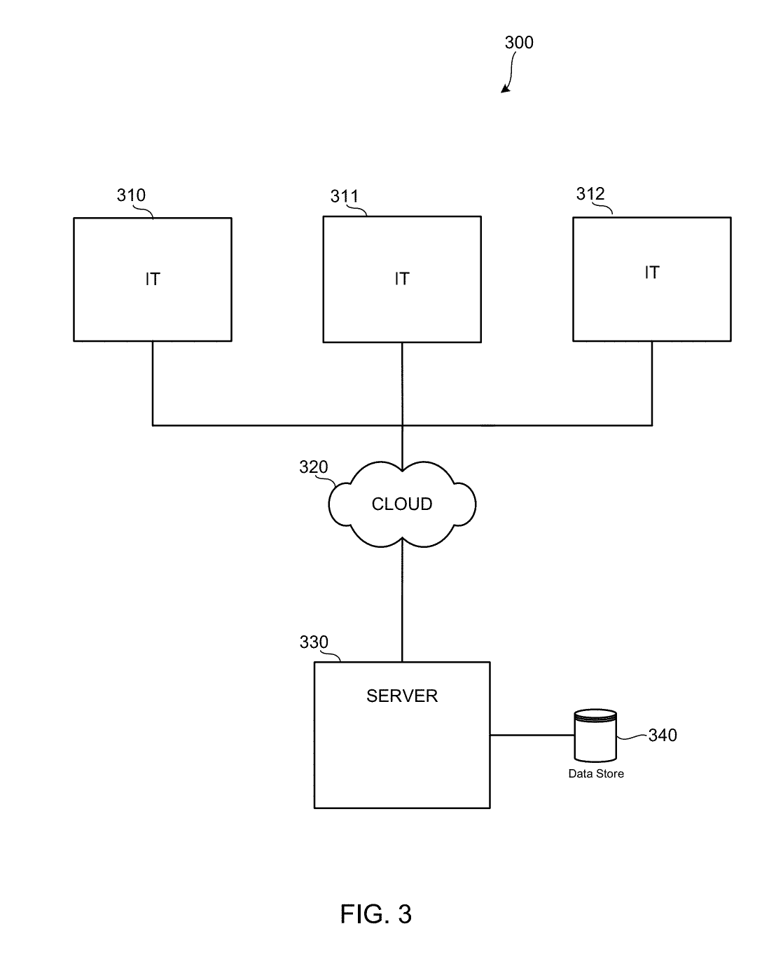

FIG. 3 illustrates an example industrial internet configuration 300. Example configuration 300 includes a plurality of health-focused systems 310-312, such as a plurality of health information systems 100 (e.g., PACS, RIS, EMR, etc.) communicating via industrial internet infrastructure 300. Example industrial internet 300 includes a plurality of health-related information systems 310-312 communicating via a cloud 320 with a server 330 and associated data store 340.

As shown in the example of FIG. 3, a plurality of devices (e.g., information systems, imaging modalities, etc.) 310-312 can access a cloud 320, which connects the devices 310-312 with a server 330 and associated data store 340. Information systems, for example, include communication interfaces to exchange information with server 330 and data store 340 via the cloud 320. Other devices, such as medical imaging scanners, patient monitors, etc., can be outfitted with sensors and communication interfaces to enable them to communicate with each other and with the server 330 via the cloud 320.

Thus, machines 310-312 within system 300 become “intelligent” as a network with advanced sensors, controls, analytical based decision support and hosting software applications. Using such an infrastructure, advanced analytics can be provided to associated data. The analytics combines physics-based analytics, predictive algorithms, automation, and deep domain expertise. Via cloud 320, devices 310-312 and associated people can be connected to support more intelligent design, operations, maintenance, and higher server quality and safety, for example.

Using the industrial internet infrastructure, for example, a proprietary machine data stream can be extracted from a device 310. Machine-based algorithms and data analysis are applied to the extracted data. Data visualization can be remote, centralized, etc. Data is then shared with authorized users, and any gathered and/or gleaned intelligence is fed back into the machines 310-312.

While progress with industrial equipment automation has been made over the last several decades, and assets have become ‘smarter,’ the intelligence of any individual asset pales in comparison to intelligence that can be gained when multiple smart devices are connected together. Aggregating data collected from or about multiple assets can enable users to improve business processes, for example by improving effectiveness of asset maintenance or improving operational performance if appropriate industrial-specific data collection and modeling technology is developed and applied.

In an example, an industrial asset can be outfitted with one or more sensors configured to monitor respective ones of an asset's operations or conditions. Data from the one or more sensors can be recorded or transmitted to a cloud-based or other remote computing environment. By bringing such data into a cloud-based computing environment, new software applications informed by industrial process, tools and know-how can be constructed, and new physics-based analytics specific to an industrial environment can be created. Insights gained through analysis of such data can lead to enhanced asset designs, or to enhanced software algorithms for operating the same or similar asset at its edge, that is, at the extremes of its expected or available operating conditions.

Systems and methods to manage industrial assets can include or can be a portion of an Industrial Internet of Things (IIoT). In an example, an IIoT connects industrial assets, such as turbines, jet engines, and locomotives, to the Internet or cloud, or to each other in some meaningful way. The systems and methods described herein can include using a “cloud” or remote or distributed computing resource or service. The cloud can be used to receive, relay, transmit, store, analyze, or otherwise process information for or about one or more industrial assets. In an example, a cloud computing system includes at least one processor circuit, at least one database, and a plurality of users or assets that are in data communication with the cloud computing system. The cloud computing system can further include or can be coupled with one or more other processor circuits or modules configured to perform a specific task, such as to perform tasks related to asset maintenance, analytics, data storage, security, or some other function.

However, the integration of industrial assets with the remote computing resources to enable the IIoT often presents technical challenges separate and distinct from the specific industry and from computer networks, generally. A given industrial asset may need to be configured with novel interfaces and communication protocols to send and receive data to and from distributed computing resources. Given industrial assets may have strict requirements for cost, weight, security, performance, signal interference, and the like such that enabling such an interface is rarely as simple as combining the industrial asset with a general purpose computing device.

To address these problems and other problems resulting from the intersection of certain industrial fields and the IIoT, embodiments may enable improved interfaces, techniques, protocols, and algorithms for facilitating communication with and configuration of industrial assets via remote computing platforms and frameworks. Improvements in this regard may relate to both improvements that address particular challenges related to particular industrial assets (e.g., improved imaging systems, medical records systems, analytics systems, etc.) that address particular problems related to use of these industrial assets with these remote computing platforms and frameworks, and also improvements that address challenges related to operation of the platform itself to provide improved mechanisms for configuration, analytics, and remote management of industrial assets.

The Predix™ platform available from GE is a novel embodiment of such Asset Management Platform (AMP) technology enabled by state of the art cutting edge tools and cloud computing techniques that enable incorporation of a manufacturer's asset knowledge with a set of development tools and best practices that enables asset users to bridge gaps between software and operations to enhance capabilities, foster innovation, and ultimately provide economic value. Through the use of such a system, a manufacturer of industrial assets can be uniquely situated to leverage its understanding of industrial assets themselves, models of such assets, and industrial operations or applications of such assets, to create new value for industrial customers through asset insights.

### D. Data Mining Examples

Imaging informatics includes determining how to tag and index a large amount of data acquired in diagnostic imaging in a logical, structured, and machine-readable format. By structuring data logically, information can be discovered and utilized by algorithms that represent clinical pathways and decision support systems. Data mining can be used to help ensure patient safety, reduce disparity in treatment, provide clinical decision support, etc. Mining both structured and unstructured data from radiology reports, as well as actual image pixel data, can be used to tag and index both imaging reports and the associated images themselves.

### E. Example Methods of Use

Clinical workflows are typically defined to include one or more steps or actions to be taken in response to one or more events and/or according to a schedule. Events may include receiving a healthcare message associated with one or more aspects of a clinical record, opening a record(s) for new patient(s), receiving a transferred patient, reviewing and reporting on an image, executing orders for specific care, signing off on orders for a discharge, and/or any other instance and/or situation that requires or dictates responsive action or processing. The actions or steps of a clinical workflow may include placing an order for one or more clinical tests, scheduling a procedure, requesting certain information to supplement a received healthcare record, retrieving additional information associated with a patient, providing instructions to a patient and/or a healthcare practitioner associated with the treatment of the patient, radiology image reading, dispatching room cleaning and/or patient transport, and/or any other action useful in processing healthcare information or causing critical path care activities to progress. The defined clinical workflows may include manual actions or steps to be taken by, for example, an administrator or practitioner, electronic actions or steps to be taken by a system or device, and/or a combination of manual and electronic action(s) or step(s). While one entity of a healthcare enterprise may define a clinical workflow for a certain event in a first manner, a second entity of the healthcare enterprise may define a clinical workflow of that event in a second, different manner. In other words, different healthcare entities may treat or respond to the same event or circumstance in different fashions. Differences in workflow approaches may arise from varying preferences, capabilities, requirements or obligations, standards, protocols, etc. among the different healthcare entities.

In certain examples, a medical exam conducted on a patient can involve review by a healthcare practitioner, such as a radiologist, to obtain, for example, diagnostic information from the exam. In a hospital setting, medical exams can be ordered for a plurality of patients, all of which require review by an examining practitioner. Each exam has associated attributes, such as a modality, a part of the human body under exam, and/or an exam priority level related to a patient criticality level. Hospital administrators, in managing distribution of exams for review by practitioners, can consider the exam attributes as well as staff availability, staff credentials, and/or institutional factors such as service level agreements and/or overhead costs.

Additional workflows can be facilitated such as bill processing, revenue cycle mgmt., population health management, patient identity, consent management, etc.

### III. Example Healthcare Gamification Systems, Dashboards, and Methods

Certain examples provide healthcare gamification systems, dashboards, and methods through a structured approach to friendly competition that drives sustained behavior change leading to improved outcomes. In certain examples, an organizational review and goal setting identifies tasks, milestones, roles, users, and other elements to drive process tracking. Based on an analysis of process tracking data, progress toward goals can be determined and displayed in conjunction with rewards, and behavior modification can be facilitated through the display as well as dynamic adjustment of tasks, milestones, roles, goals, etc., driven by the process tracking and analytics, for example.

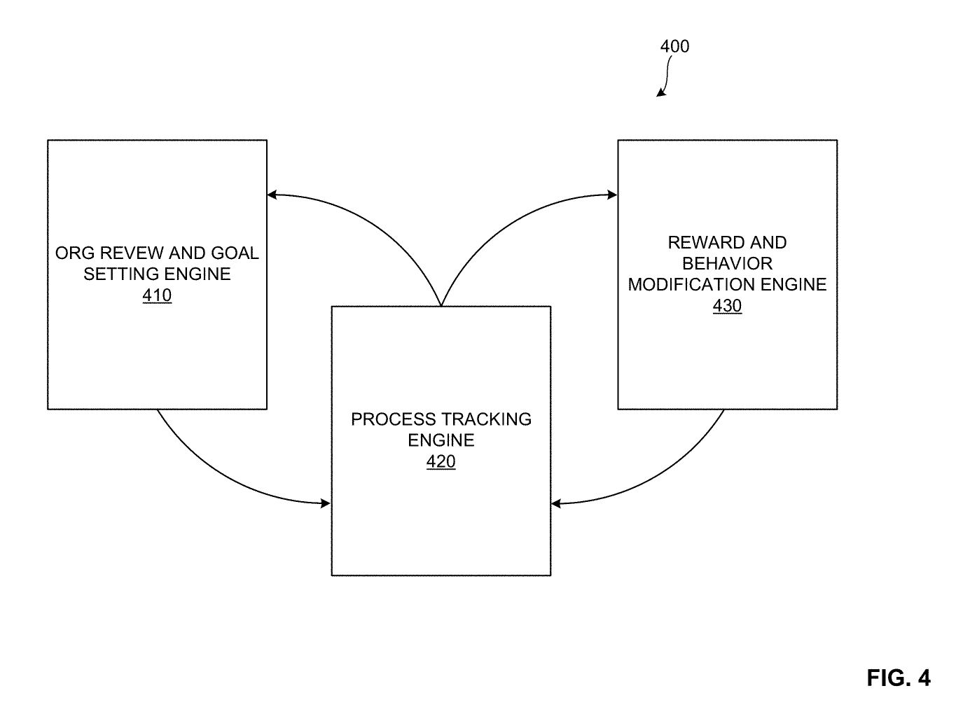

FIG. 4 illustrates an example healthcare gamification system 400 including an organizational review and goal setting engine 410, a process tracking engine 420, and a reward and behavior modification engine 430. The example organization review and goal setting engine 410 receives and processes information regarding a healthcare organization (e.g., a hospital, a hospital network, a clinic, a doctor's office, etc.) and the organization's workflows, goals, rules, users, roles, etc. The example engine 410 generates new workflows, organizational goals, reward rules, weights, values, etc. For example, the engine 410 identifies one or more applicable workflows, processes the workflow(s), and applies game rules to each application workflow.

The example process tracking engine 420 receives information such as workflows, goals, reward rules, weights, and values, etc., from the setting engine 410 and uses the information to define processes and associated tasks based on the workflows, etc. The process tracking engine 410 can couple goals and rewards with historical data to apply a score for each user based on organizational goals, for example. The process tracking engine 420 monitors/tracks ongoing processes for the organization and provides feedback, such as tracking data and/or associated score, to the organizational review and goal setting engine 410 for retroactive review, etc. Such feedback can be used by the engine 410 to define new workflow(s), goal(s), reward rule(s)/weight(s)/value(s), etc. The process tracking engine 420 can also provide tracking data to the rewards and behavior modification engine 430 for personal review, etc.

The example reward and behavior modification engine 430 processes tracking data from the process tracking engine 420 and generates personal goals for one or more users, group of users, etc. For example, the engine 430 defines goal(s) and reward(s) per the identified workflow(s). The goal(s) can be fed back into the process tracking engine 420 for monitoring and feedback to the engines 410, 430, etc.

Via the example system 400, managers and healthcare organization leaders can set and change goals, rewards, workflow associations, etc. Via the engine 410, workflow data from a health information system (e.g., Centricity® Business, etc.) is read in and processed for healthcare insight. The engine 410 processes the input data and associated generated insights and correlates the information with a reward table. After the reward table information and the healthcare data are coupled together, the review and goal setting engine 410 generates information and dashboard(s) such as gamer access, leader board(s), user board(s), and post case analytics, etc., and makes the information and dashboard interface(s) available for review, interaction, further processing, etc.

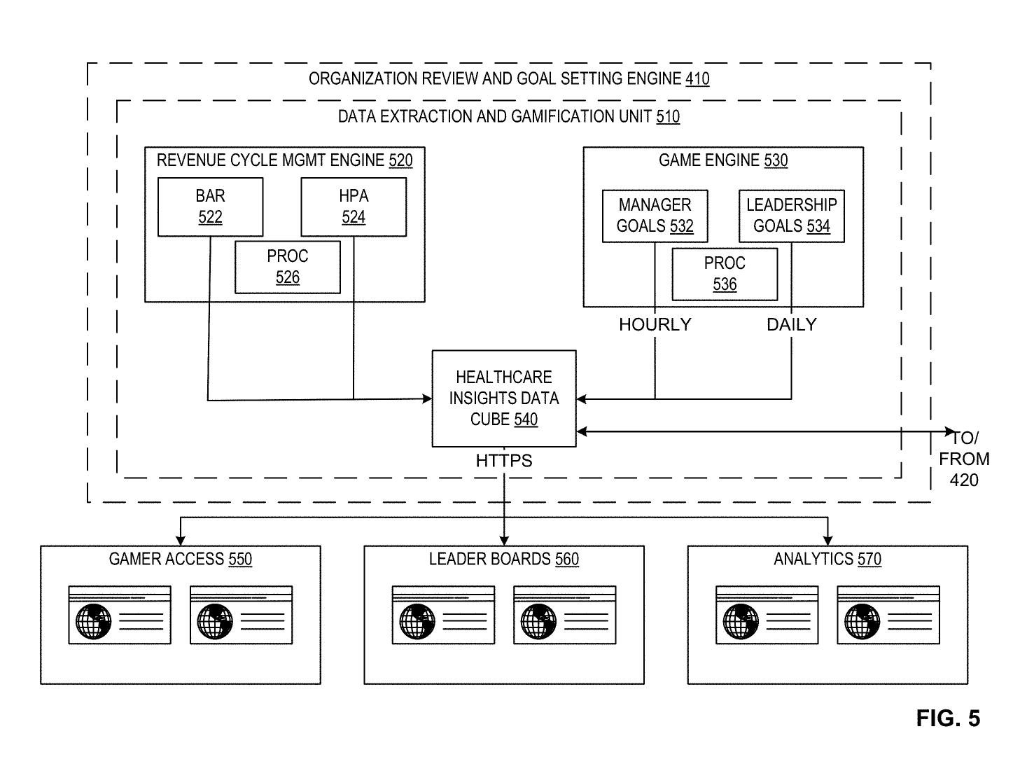

FIG. 5 illustrates an example implementation of the organization review and goal setting engine 410 of FIG. 4. In the example of FIG. 5, a data extraction and gamification unit 510 in the engine 410 processes and gamifies healthcare data. The example unit 510 includes a revenue cycle management engine 520 and a game engine 530.

The revenue cycle management engine 520 includes a billing and accounts receivable (BAR) engine 522, a hospital patient accounting (HPA) engine 524, and a processor 526. The BAR engine 522 leverages the processor 526 and provides, processes, and tracks billing information and billing workflow information including outgoing invoices, received payments, etc., for example. The HPA engine 524 leverages the processor and provides, processes, and tracks patient charges, billing, and receivables, for example. The HPA engine 524 works with the BAR engine 522 and processor 526 and facilitates the patient workflow for a healthcare organization, for example. Using the HPA engine 524, charges, payments, debit and credit adjustments, etc., are created upon patient visits. When the visits are ready to be billed, claims and patient bills are produced and reconciled with the BAR engine 522. The HPA engine 524 can alert users when additional data is warranted, when incorrect data has been entered, etc. The BAR engine 522 can alert users when payments are made, payments are delinquent, etc. Feedback from the BAR engine 522 and the HPA engine 524 works with the game engine 530 to generates dashboard interface(s), progress toward goal(s), reward(s), etc. Using the HPA engine 524 and/or the BAR engine 522, operation and managerial reports and analyses can be produced to monitor workflows, goals, progress, etc.

The game engine 530 includes manager goals 532, leadership goals 534, and a processor 536. In the example of FIG. 5, manager goals 532 are updated hourly via the processor 536, and leadership goals 534 are updated daily via the processor 536, although the frequency can vary. The game engine 530 processor 536 processes and provides those goals and accepts input from user(s) to set and/or change such goals 532 and/or 534. The game engine 530 processor 536 applies game rules to healthcare data to gamify associated workflow(s), tasks(s), goal(s), etc.

The revenue cycle management engine 520 and the game engine 530 provide output to a healthcare insights data cube 540. The data cube 540 forms and stores insights to the healthcare data from the combination of revenue cycle (e.g., BAR, HPA, etc.) data with gamification data, for example. Insights and other information from the data cube 540 are transmitted and used to form a plurality of dashboard interfaces 550, 560, 570.

As shown in the example of FIG. 5, one or more gamer access or user boards 550 can be provided to show individual user (e.g., healthcare gamer) information such as goals, progress toward goals, badges, rewards, rank, etc. Additionally, one or more leader boards 560 can be provided to show ranking(s) of users according to goals, rules, progress, etc. Further, one or more analytics dashboards 570 can provide additional analytics, metrics, data breakdowns, etc., based on the gamification and analysis of revenue cycle management and other healthcare data according to workflow, organization, and game rules.

Thus, the revenue cycle management engine 520 and game engine 530 repeatedly (e.g., continuously, periodically, on-demand, as triggered by an event, etc.) extract data from healthcare workflow applications. These extracts are then moved into a centralized location (e.g., the organization review and goal setting engine 410) where they can then be loaded into an optimized analytics data structure, such as the healthcare insights data cube 540. The dashboards 550, 560, 570 are connected into the analytics data structure(s) 540 to provide end user visualizations of the data. In certain examples, the game engine 530 uses a reward table as a meta-data table to derive points or coins incremented or decremented against a number of records in each user's task activity. This allows the management team to change the meta data, which impacts the scoring system. Managers can manipulate this data to drive desired behavior characteristics of the staff, for example. In certain examples, visualizations are delivered over a secure web uniform resource locator (URL) and allow for filtering, drilling into detail data (if enabled) and exporting of widgets and/or the whole dashboard 550, 560, 570 into an image file, pdf, and/or the underlying data in comma separated values (.csv format), etc.

For example, the data cube 540 conducts periodic builds (e.g., hourly, daily, on demand, etc.) to calculate scores/opportunities per user/gamer. Game play does not affect a transactional database storing clinical and/or operational data, for example. Data, such as BAR 522, HPA 524, etc., are periodically extracted (e.g., hourly, daily, on demand, etc.) and provided with updated data from gamer/user actions within a clinical application to enable gameplay on enterprise task manager (ETM) processes, for example. The game engine 530 can provide updated leadership goals 534 (e.g., daily, on demand, etc.) including new weights per outcome, bonus options for users/teams, etc. The game engine 530 can also provide updated manager goals 532 (e.g., hourly, daily, on demand, etc.) including bonus options for users, awards/badges, etc.

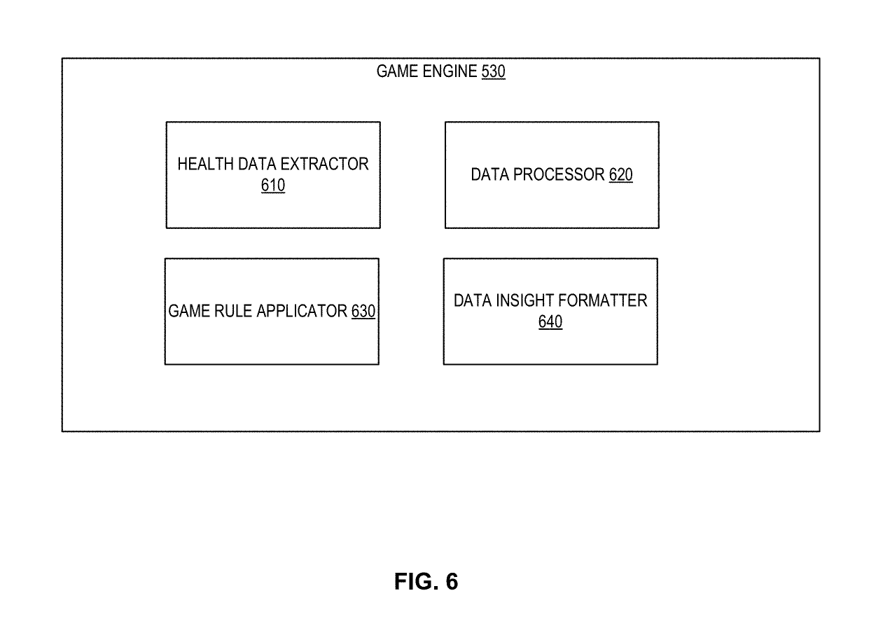

FIG. 6 illustrates an example implementation of the game engine 530 of the example of FIG. 5. As shown in the example of FIG. 6, the game engine 530 includes a healthcare data extractor 610, a data processor 620, a game rule applicator 630, and a data insight formatter 640. The example data extractor 610 extracts (e.g., continuously, periodically, on-demand, as triggered by an event, etc.) data from one or more healthcare applications executing at and/or associated with the healthcare organization. Extracted data is processed by the data processor 620 (e.g., to format the data, remove data outside a certain threshold, range, criterion, etc.).

The example game rule applicator 630 then applies game rule(s) to the processed data to evaluate progress toward goals, reward badges, generate comparative rank, etc. The game rule applicator 630 can leverage a rewards table and its meta-data to derive points, coins, badges, etc., incremented or decremented against a number of records in each user's task activity. Table meta-data and input healthcare data can be changed, manipulated, etc., to adjust scoring and outcomes and drive desired behavior characteristics of users, for example.

The example data insight formatter 640 takes the data and gamification results and formats the data for healthcare insight analysis and use in dashboard interfaces, etc. The data insight formatter 640 then provides the formatted data for storage in an analytics data structure at the healthcare insights data cube 540. The dashboards 550, 560, 570 are connected into the analytics data structure(s) 540 to provide end user visualizations of the data.

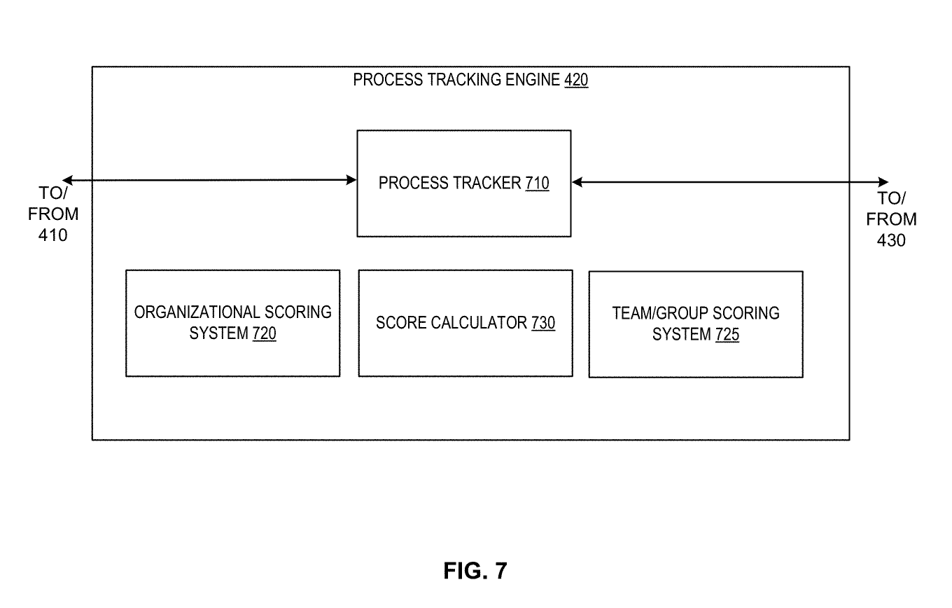

FIG. 7 illustrates an example implementation of the process tracking engine 420 of FIG. 4. The example processing tracking engine 420 includes a process tracker 710, a plurality of scoring systems 720-725, and a score calculator 730. The process tracker 710 receives information from the organization review and goal setting engine 410 including goals 532, 534, rankings, access information, analytics, rules, weights, values, workflows, etc. The process tracker 710 updates workflow(s) based goals 532 and/or 534, leader board 560 information, gamer access 550, analytics, 570, etc.

The example engine 420 can include a plurality of scoring systems 720-275 such as organizational scoring system(s) 720, group/team scoring system(s) 725, etc. Each scoring system 720, 725 can provide a score/rank such as a starting point score rank and/or output score rank, etc., to the score calculator 730. For example, the organizational scoring system 720 can generate a starting point score based on organizational leadership goals 534, etc., and the group/team scoring system 725 can generate a starting point score based on team/group manager goals 532, etc. The score/rank information is fed into the score calculator 730 for per task calculation, user score, etc. Such score information can be fed back into the user board(s) 550, leader board(s) 560, analytics 570, etc.

The example score calculator 730 performs a per task calculation and a user score based on input information from the process tracker 710, organization scoring system 720, group/team scoring system 725, and/or other input from the engine 410, etc. The score calculator 730 evaluates as tasks are completed to determine a per task score calculation. As shown in Equation 1, the score calculator 730 takes a starting point score/rank from the organizational scoring system 720 and/or the team/group scoring system 725 and also evaluates an output score/rank upon task completion from the organizational scoring system 720 and/or the team/group scoring system 725 to determine score (e.g., task coins, etc.) earned for that task:

Starting Point Score/Rank×Output Score/Rank=Task Score (Eq. 1).

As management and teams update their corresponding scoring system 720-725, the associated task is associated with the new goals and score of the newly updated management goals 532, etc.

The score calculator 730 can then calculate a user's score by adding the computed task score to a baseline and/or other existing score for the user and/or group of users. As shown in Equation 2, a total score can be determined by the example score calculator 730, and any additional points (e.g., power ups and/or other earned and/or arbitrary points/coins) awarded by team and/or management can also be added to impact the user's total score:

Task Score+Base Line Score(+Additional Points)=Total Score

The total score can be evaluated to generate and/or update the user board 550, leader board 560, analytics 570, etc. The score calculator 730 can provide score information to the process tracker 710 to be provided back to the example engine 410 and/or the example rewards and behavior modification engine 430, etc.

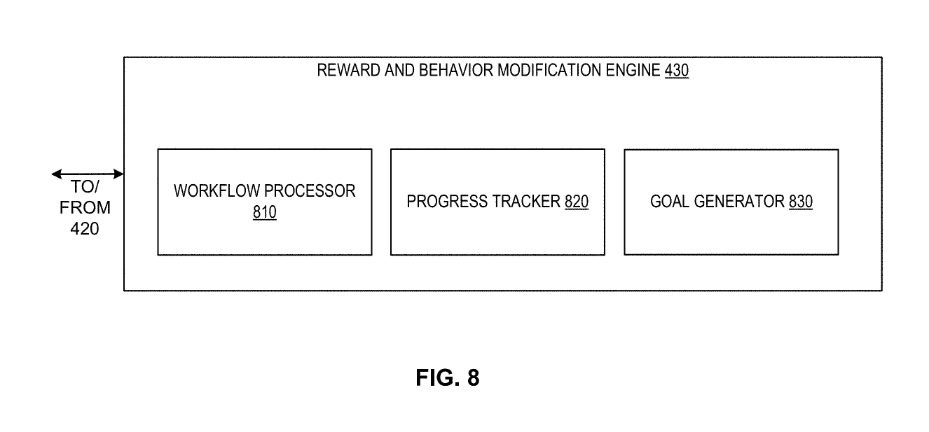

FIG. 8 illustrates an example implementation of the example reward and behavior modification engine 430 of FIG. 4. The example engine 430 includes a workflow processor 810, a progress tracker 820, and a personal goal generator 830. The example workflow processor 810 processes tracking data from the process tracking engine 420 (e.g., including workflow data routed from the organizational review and goal setting engine 410, etc.) and breaks down the workflow into tasks and associated scores. The example progress tracker 820 tracks progress toward task completion, workflow completion, score goal(s), etc. The example goal generator 830 generates personal goals for one or more users, group of users, etc. For example, the goal generator 830 defines goal(s) and reward(s) per the identified workflow(s). The goal(s) can be fed back into the process tracking engine 420 for monitoring and feedback to the engines 410, 430, etc.

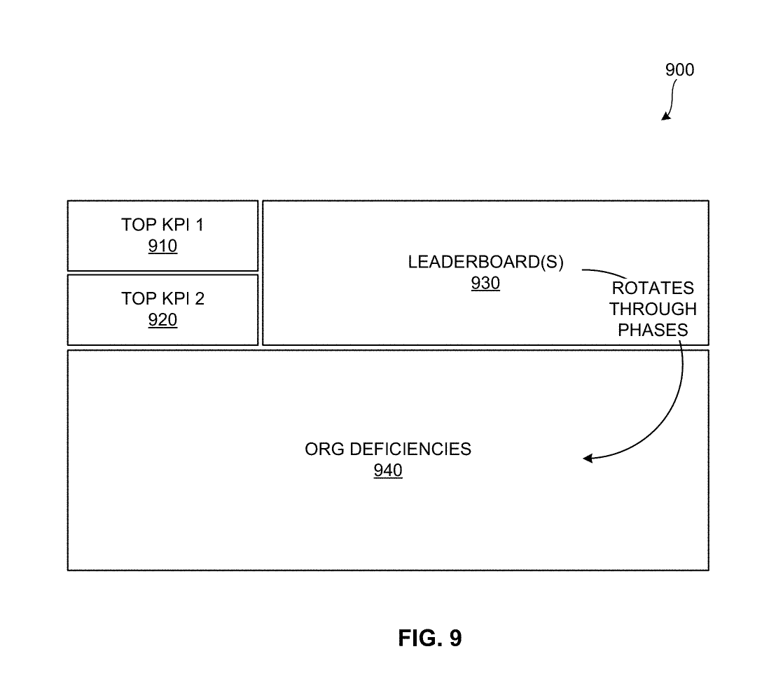

FIG. 9 illustrates an example group view leaderboard tracker 900. The example leaderboard tracker 900 includes a first key performance indicator (KPI) segment 910 to visualize and monitor a top KPI, such as cash tracked versus a goal. The example tracker 900 includes a second KPI segment 920, such as cash tracked versus a goal. Additional areas 930, 940 of the tracker dashboard 900 include a leaderboard 930 and an organization deficiency 940. As shown in the example of FIG. 9, the areas 930, 940 can rotate through various phases to show a variety of information. Thus, using the example tracker 900, daily goals and rewards can be visualized and progress by users can be monitored to evaluate improvement in productivity and task completion, for example.

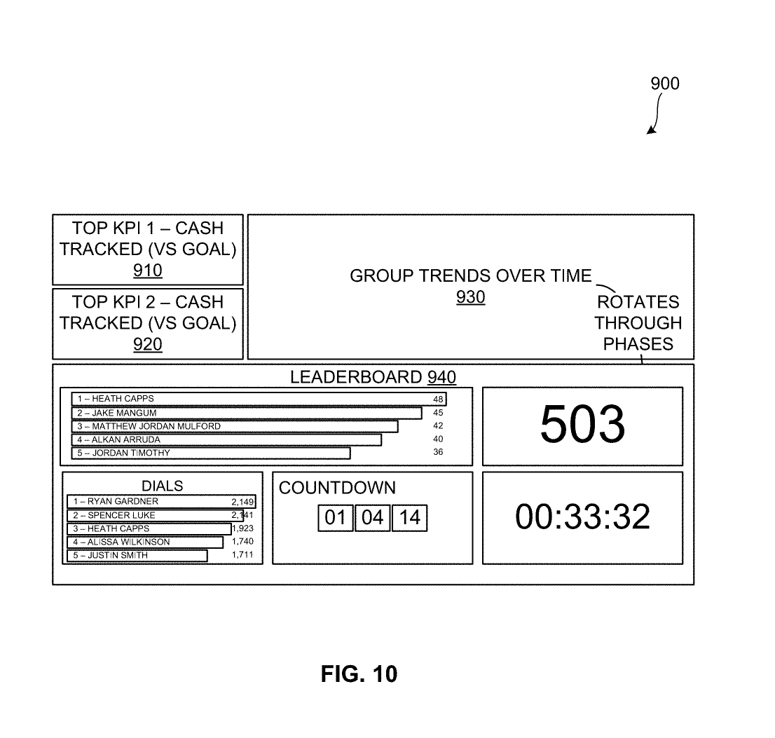

FIG. 10 illustrates a particular example implementation of the tracker 900 showing cash tracked versus goal and group trends over time for a group of leading users along with scores and a countdown to completion, etc.

The example leaderboard visualizations of FIGS. 9-10 are based on information derived from a target healthcare workflow in combination with a reward table. The leaderboard 900 is where a user can validate that their efforts have been achieved and compare themselves against their peers.

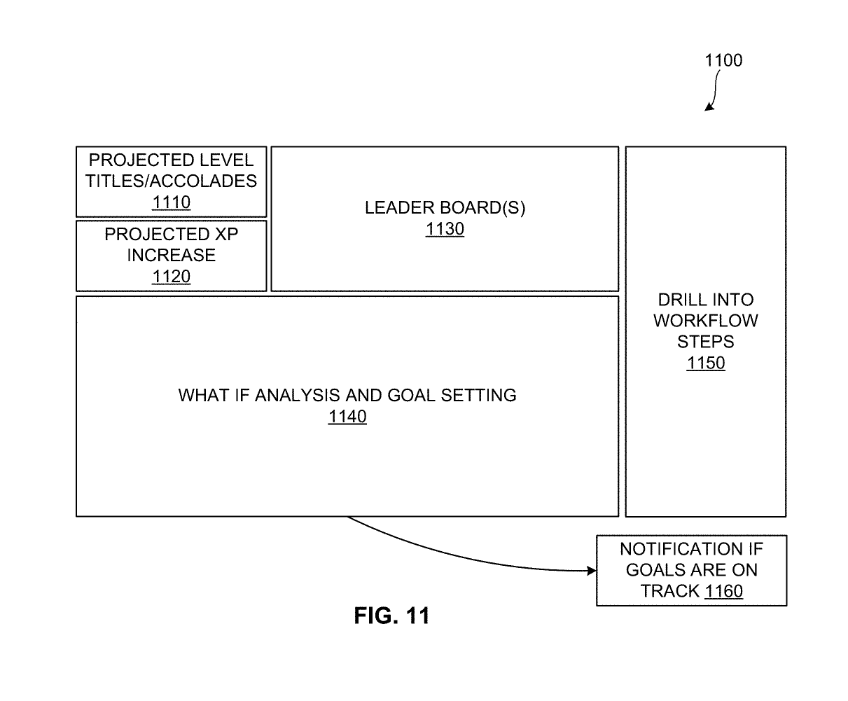

FIG. 11 illustrates an example end user dashboard 1100. The example dashboard interface 1100 is an individual user view showing their progress as well as a private location to view progress against team members, coworkers, etc. As shown in the example of FIG. 11, the dashboard interface 1100 includes projected accolades 1110, projected experience points (XP) increase 1120, leader board(s) 1130, what-if analysis and goal setting 1140, and a workflow area 1150. The scenario/simulation (e.g., what-if) analysis and goal setting portion 1140 of the interface 1100 can trigger a notification 1160 regarding whether goal(s) are on track for that user. The workflow area 1150 allows the user to drill down into particular workflow step(s) and view, verify, and/or otherwise monitor progress for particular tasks in the workflow, for example.

Thus, certain examples provide continuous feedback for delayed and real-time (or substantially real-time given some data transmission, processing, and/or storage delay) access to historical workflow data and updated reward table. Certain examples provide minimally invasive feedback including a leaderboard for shared access. Leaderboard(s) can be updated on a timeframe, rhythm, and/or trigger and/or real-time, etc. Certain examples provide team and individual goal setting, as well as organizational goal setting for leadership to steer organizational behavior, etc. Certain examples facilitate rewarding of users.

While traditional analytics provide reporting and business intelligence with visualization, no prior solution combines source system data, analytics, rules, and rewards with healthcare related tasks. Certain examples couple workflow with analytics to derive a gamification solution.

Certain examples provide a technological improvement through a system that enables manipulation of a points scoring system to drive end-user behavior via gamification of healthcare workflows, and provides visualization subsets in one or more dashboards.

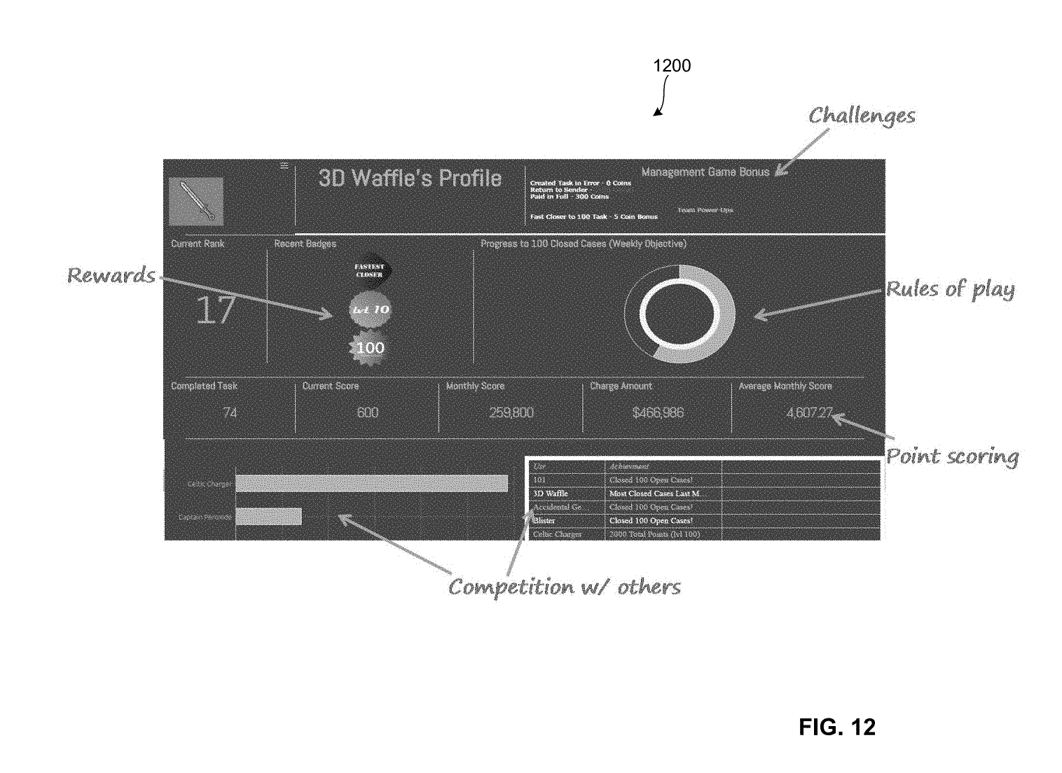

FIG. 12 illustrates an example user board 1200 including an avatar, a user profile indicator, an indication of challenges, rank, badges earned, rules, progress to completion, tasks completed, points scored, competition with others, etc.

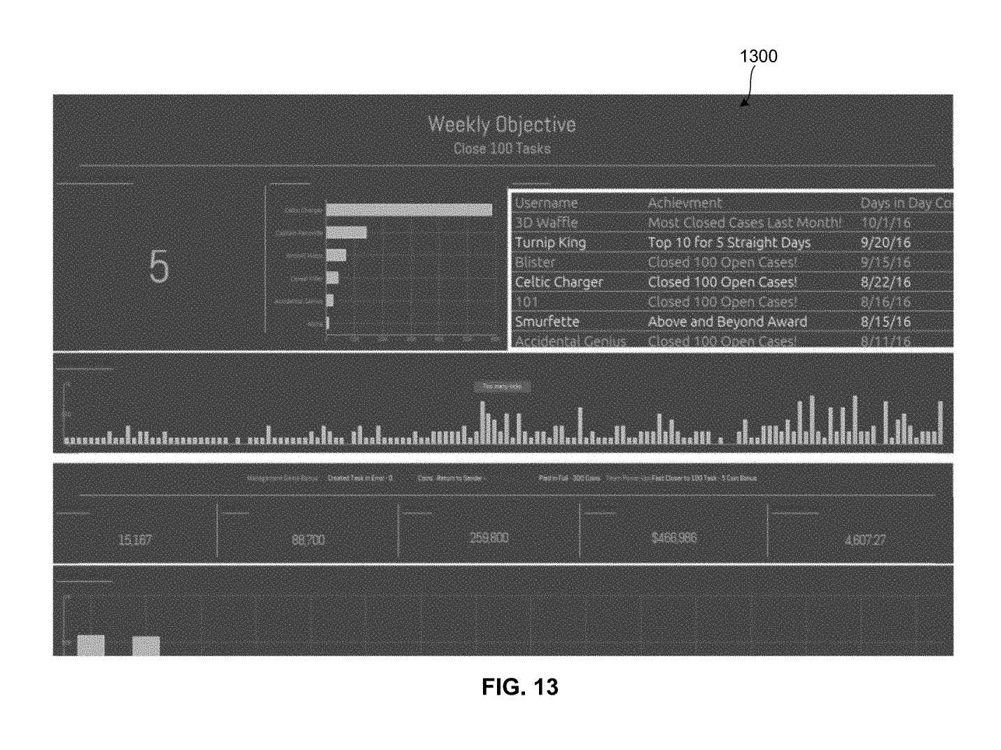

FIG. 13 illustrates an example dashboard 1300 showing weekly objectives including task, user, achievement, timeframe, dollars/coins earned, etc.

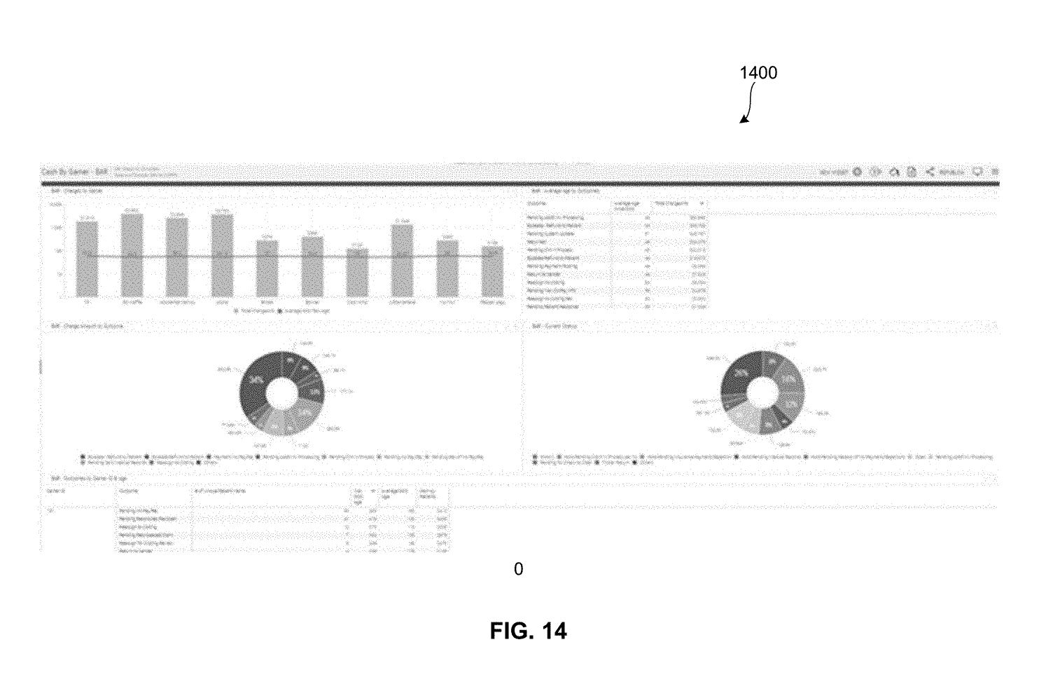

FIG. 14 illustrates an example interface 1400 showing cash by gamer in bar chart, pie chart, and alphanumeric data format. The example interface 1400 allows viewing and adjustment of progress toward billing and collection of accounts receivable, etc., for example.

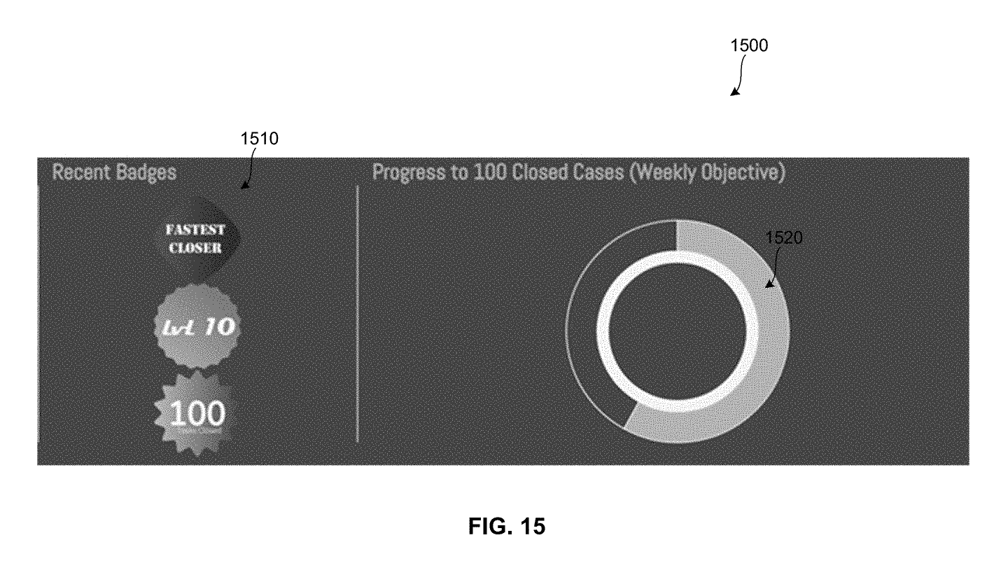

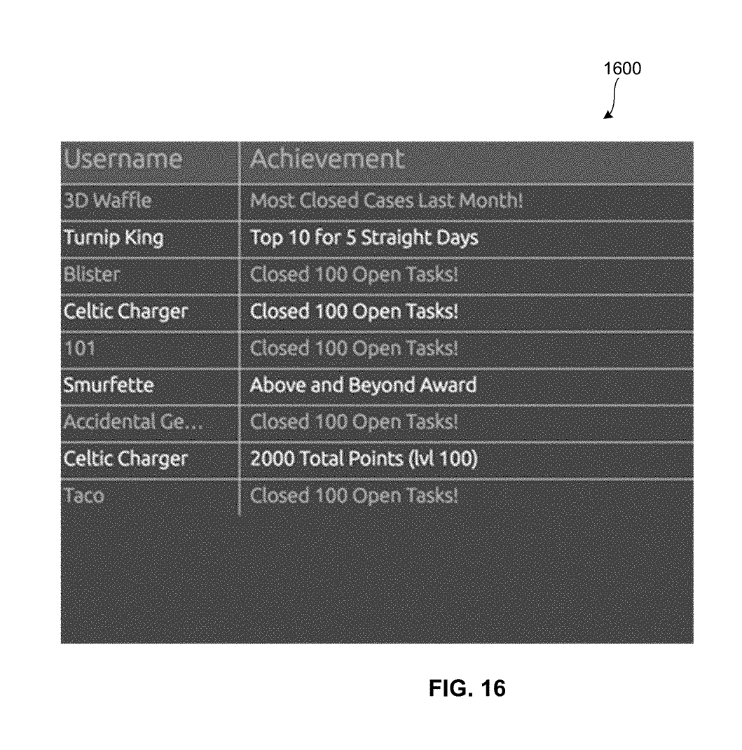

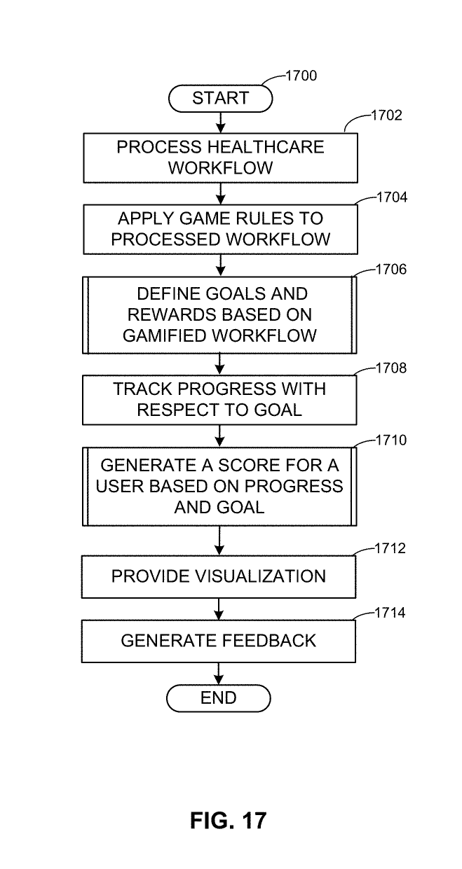

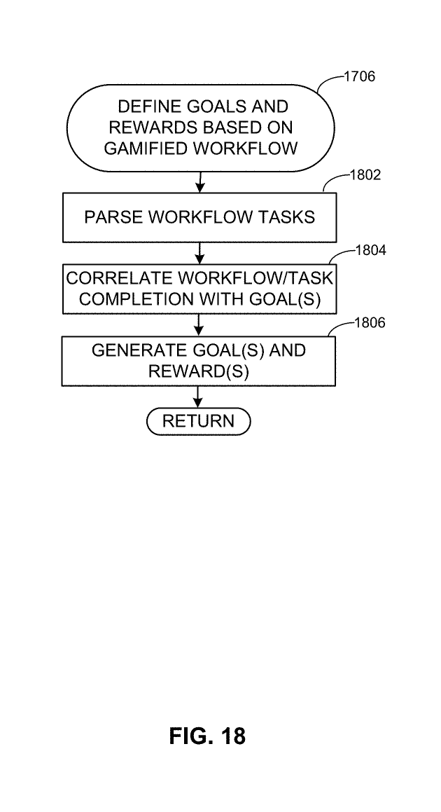

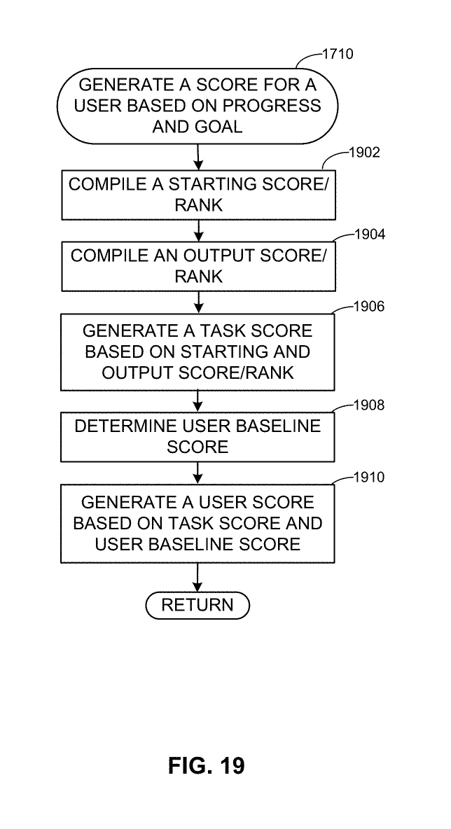

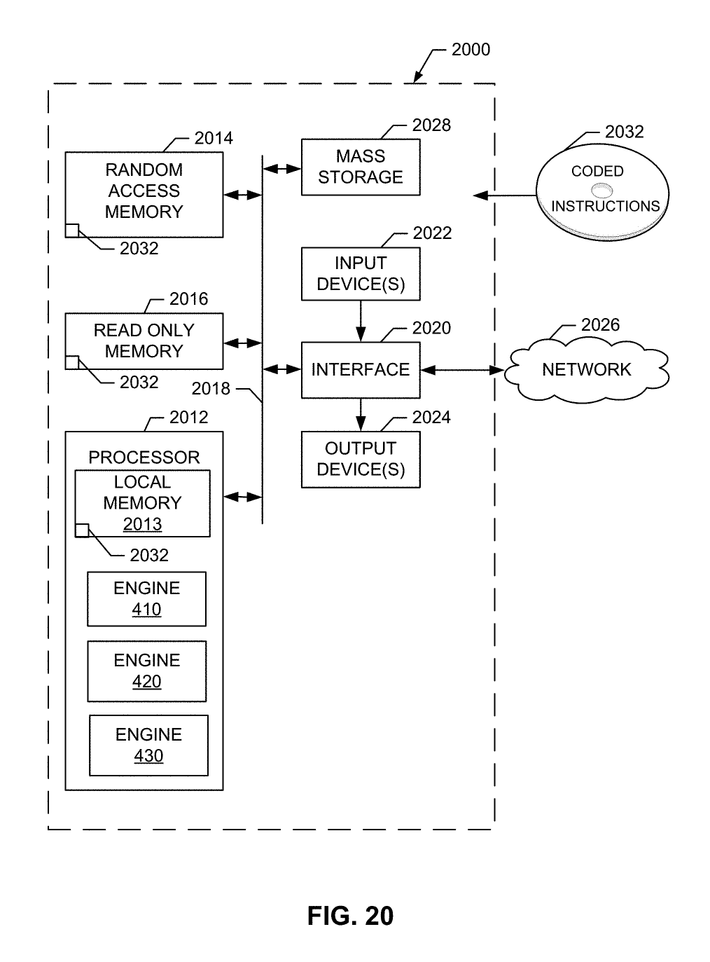

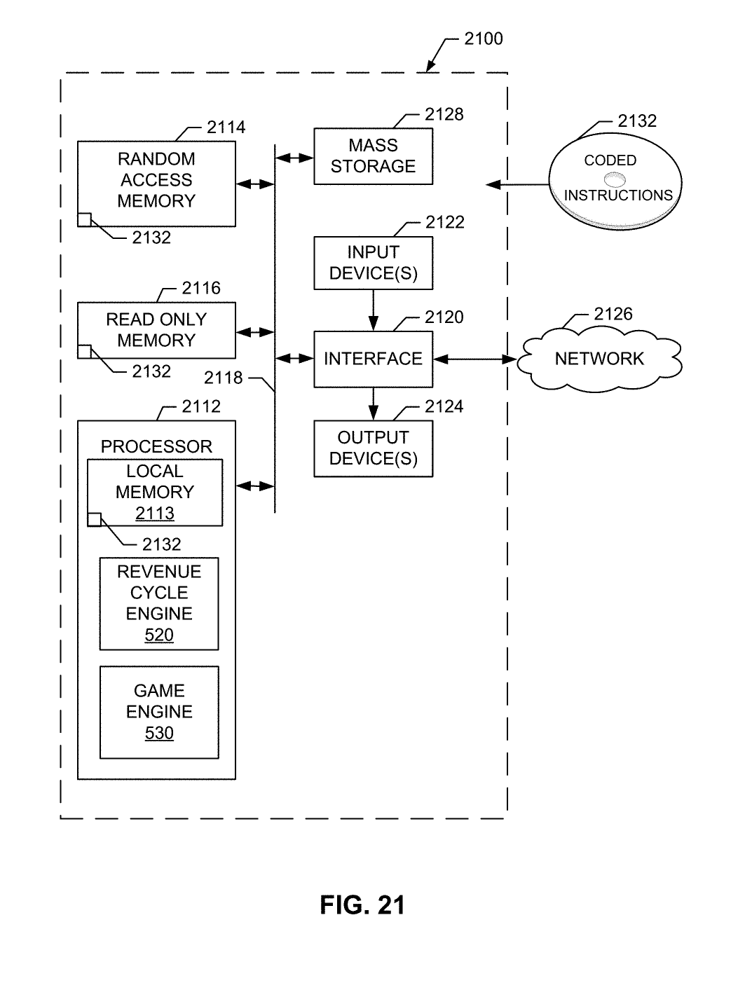

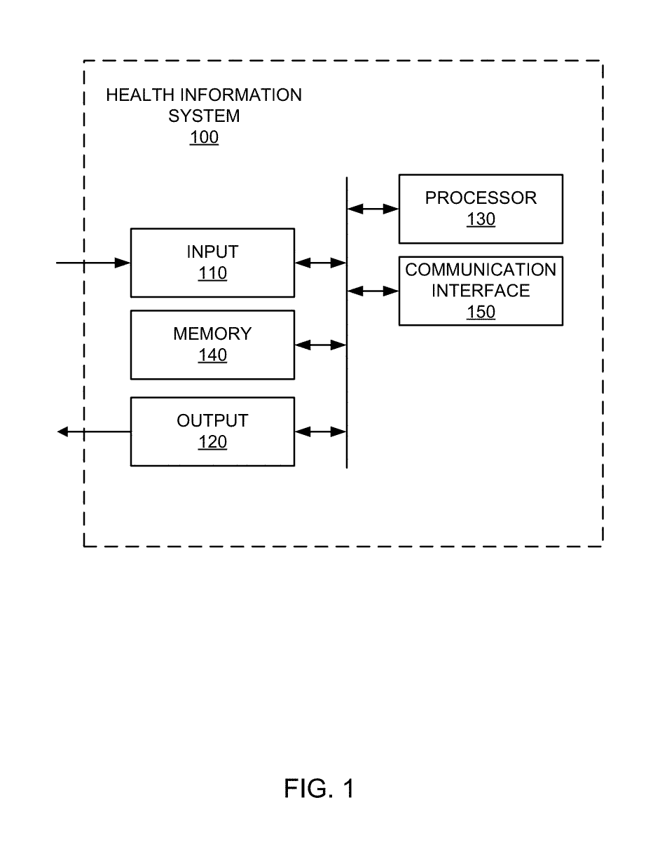

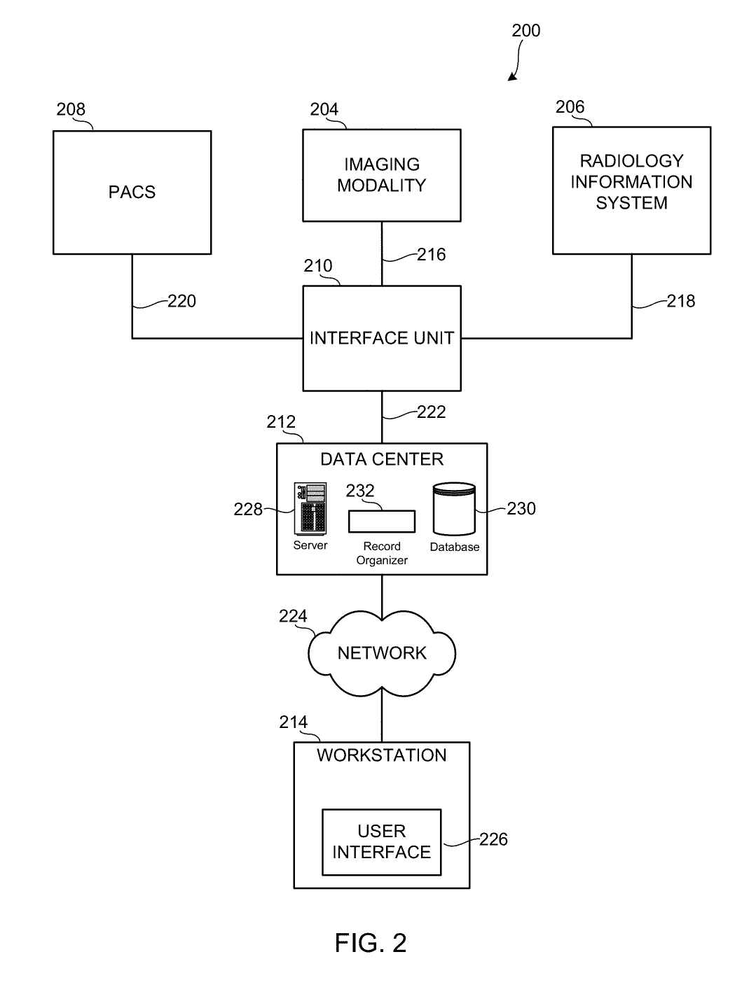

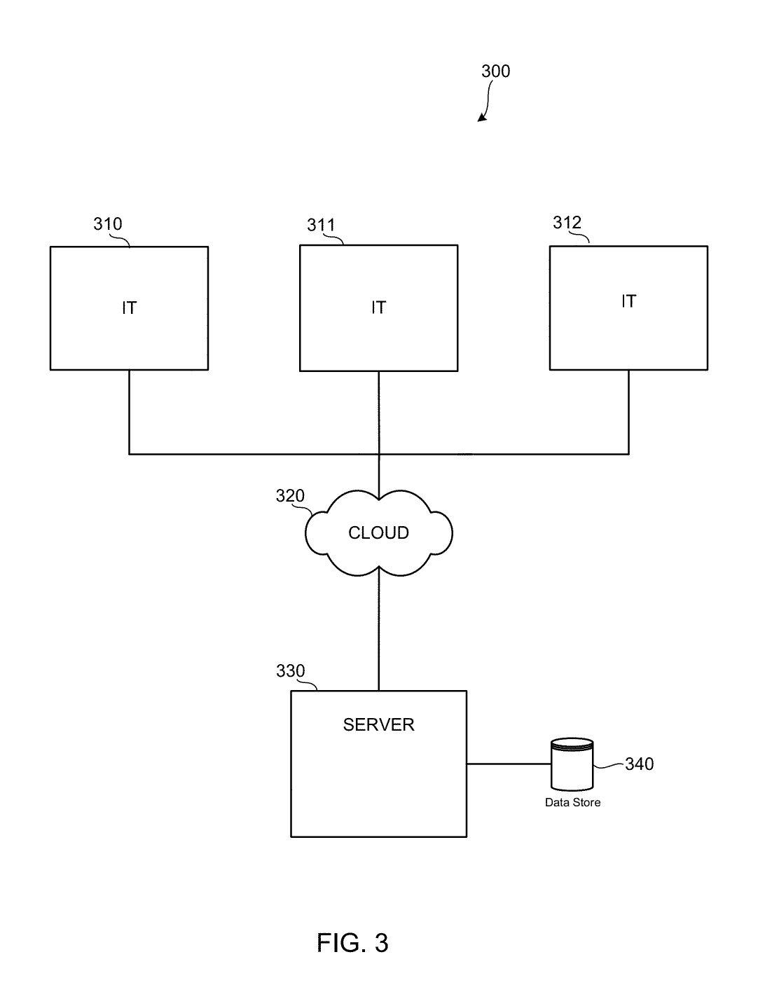

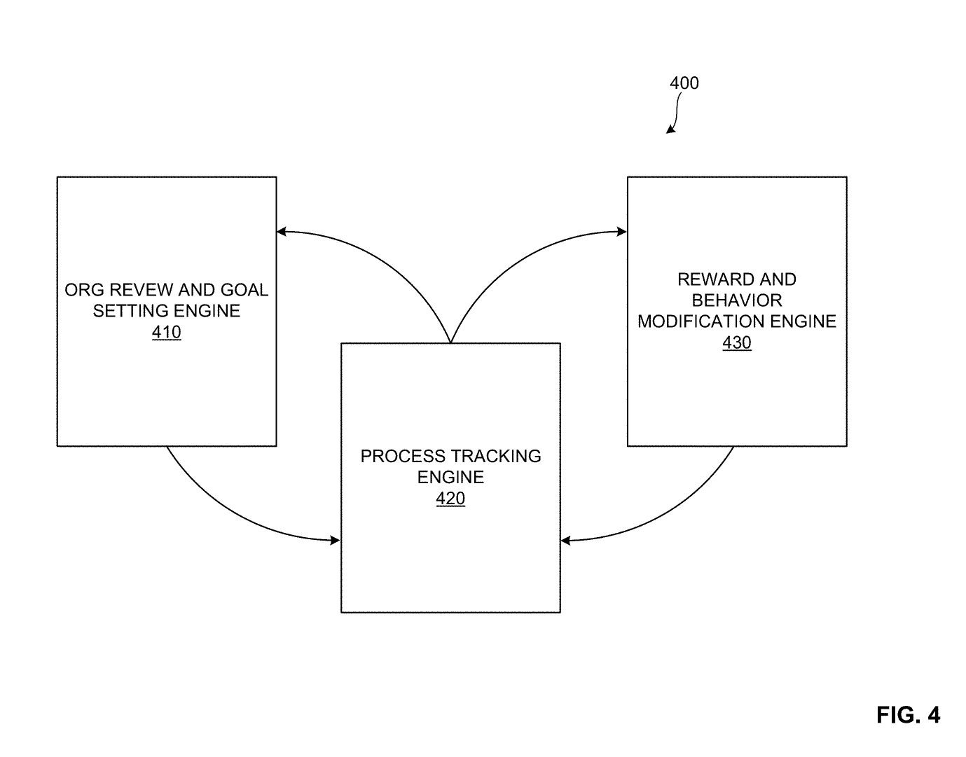

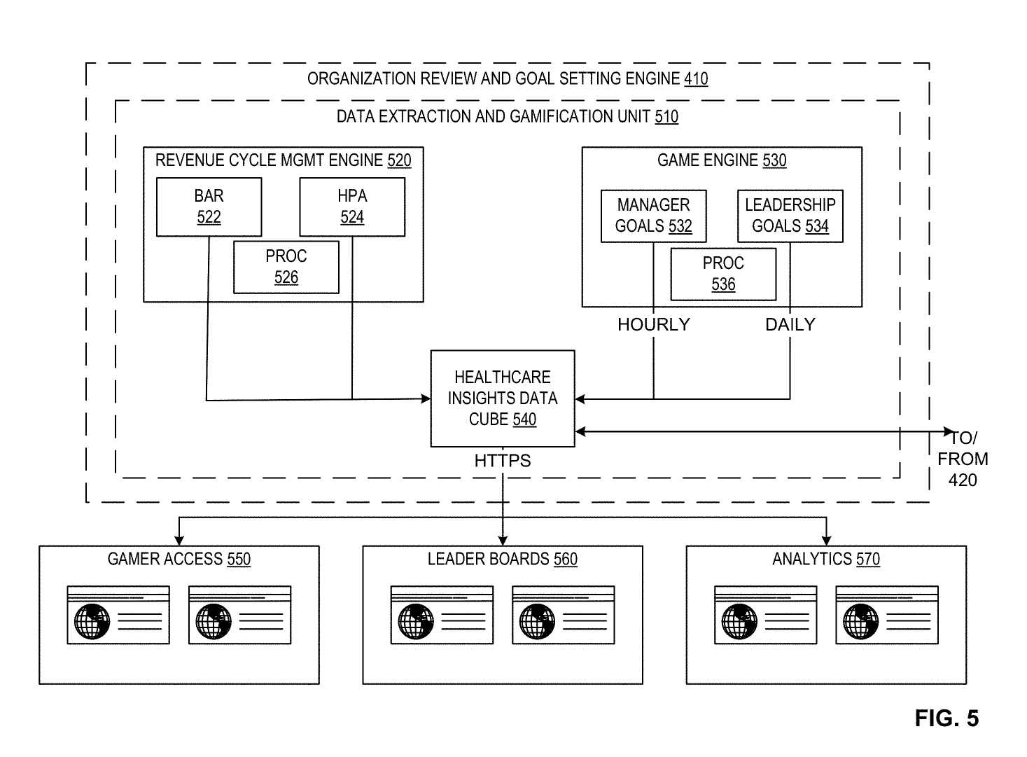

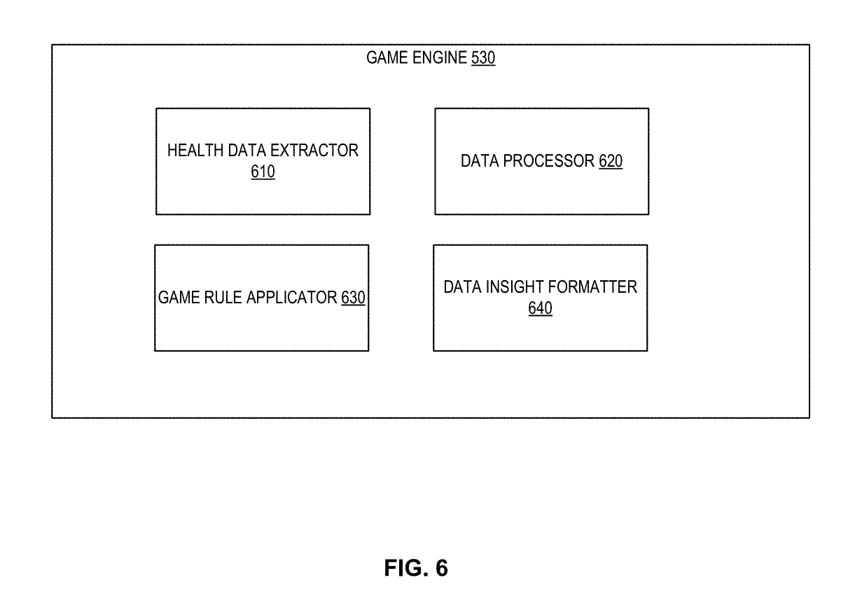

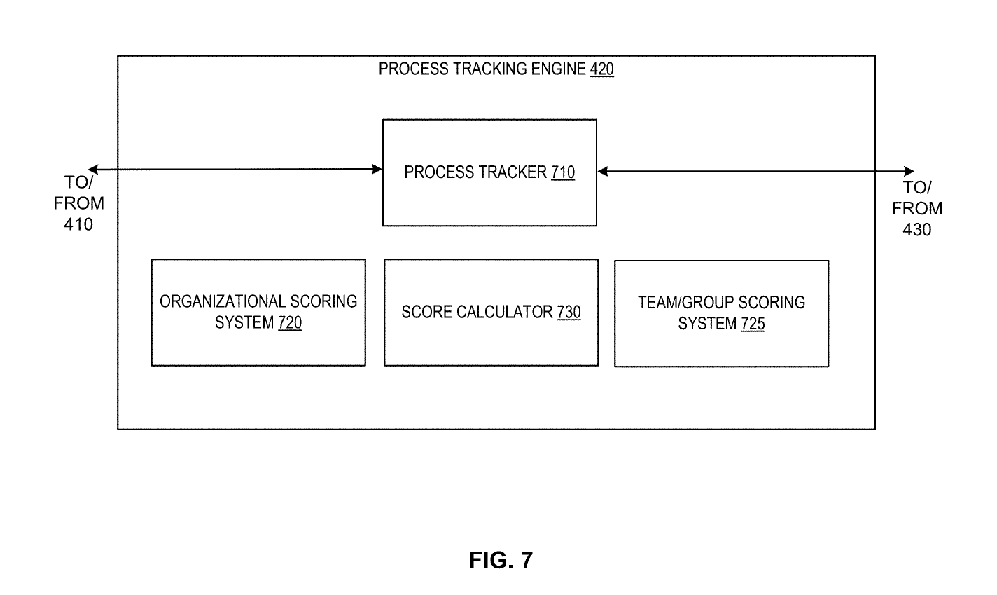

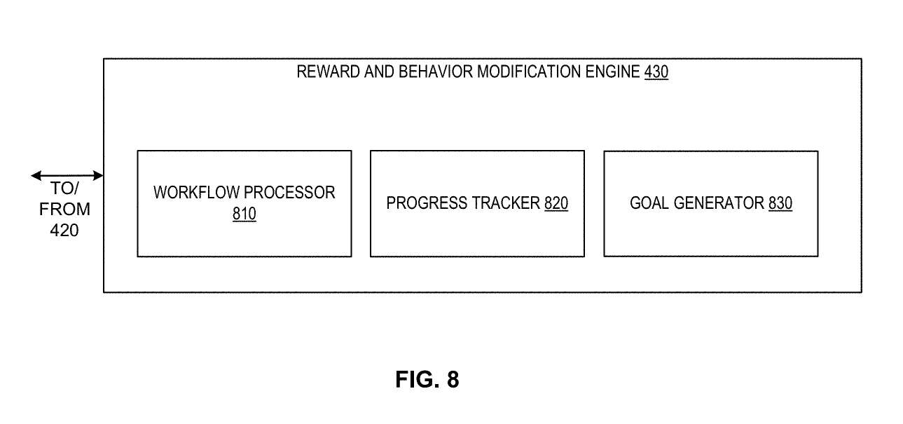

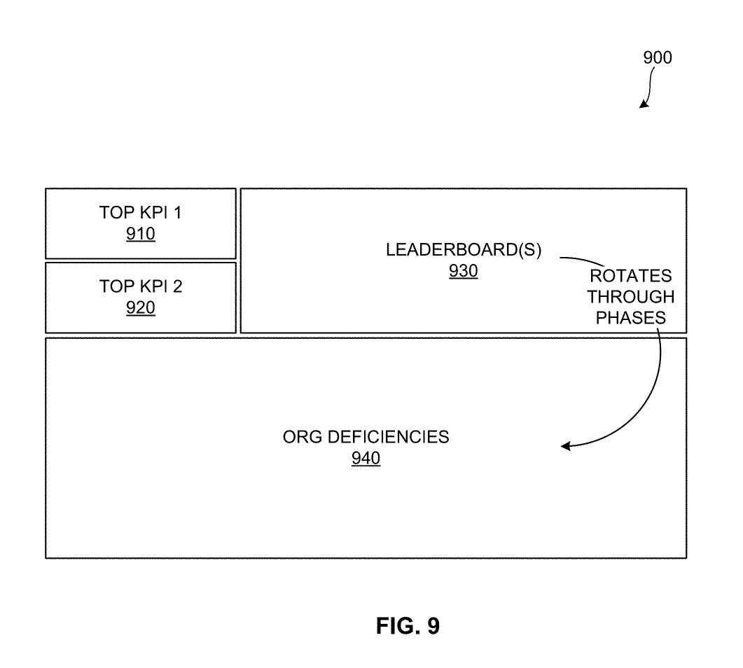

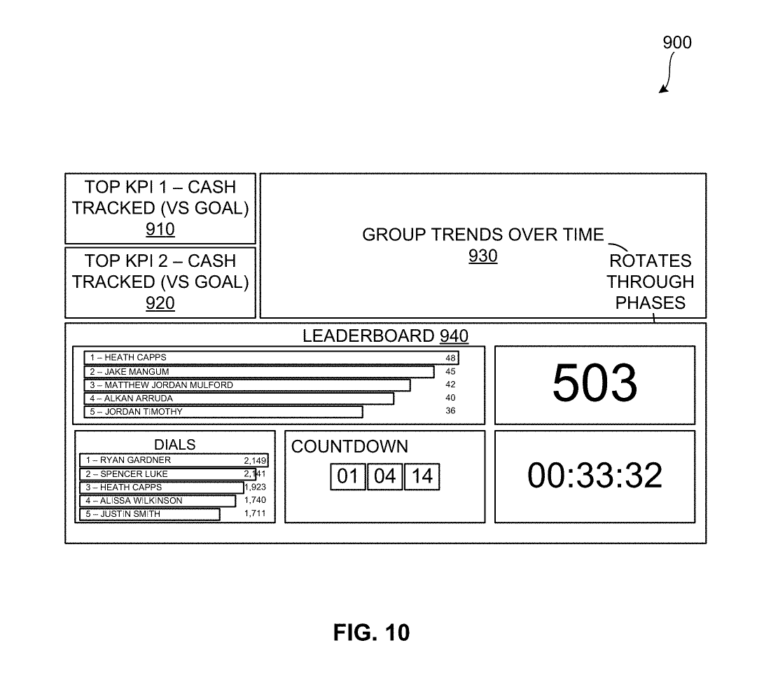

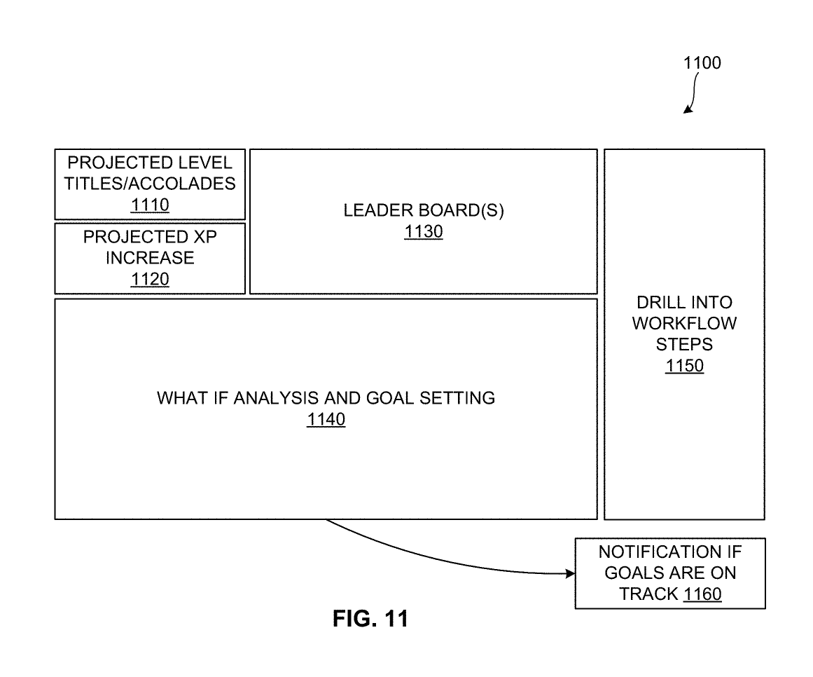

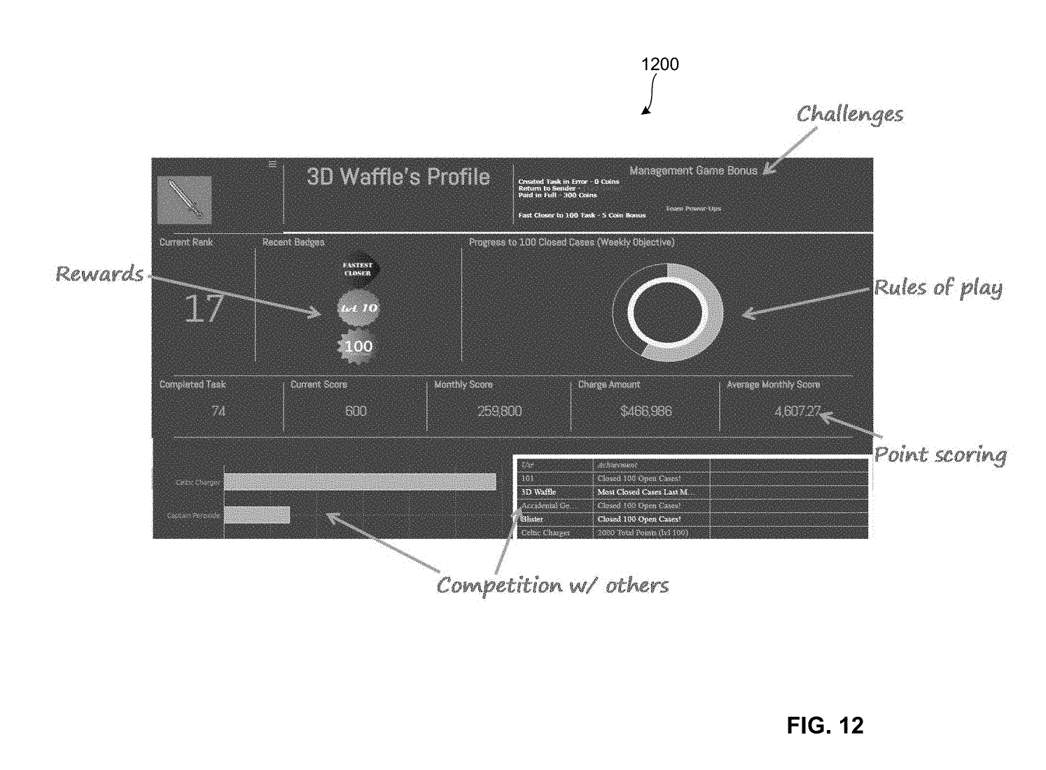

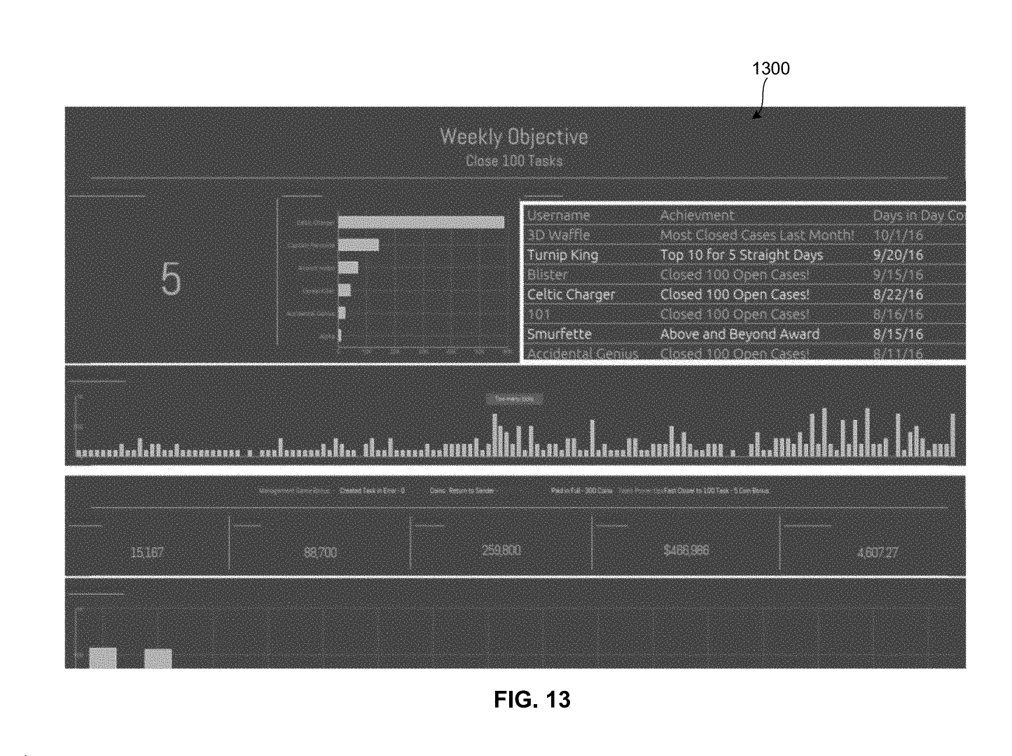

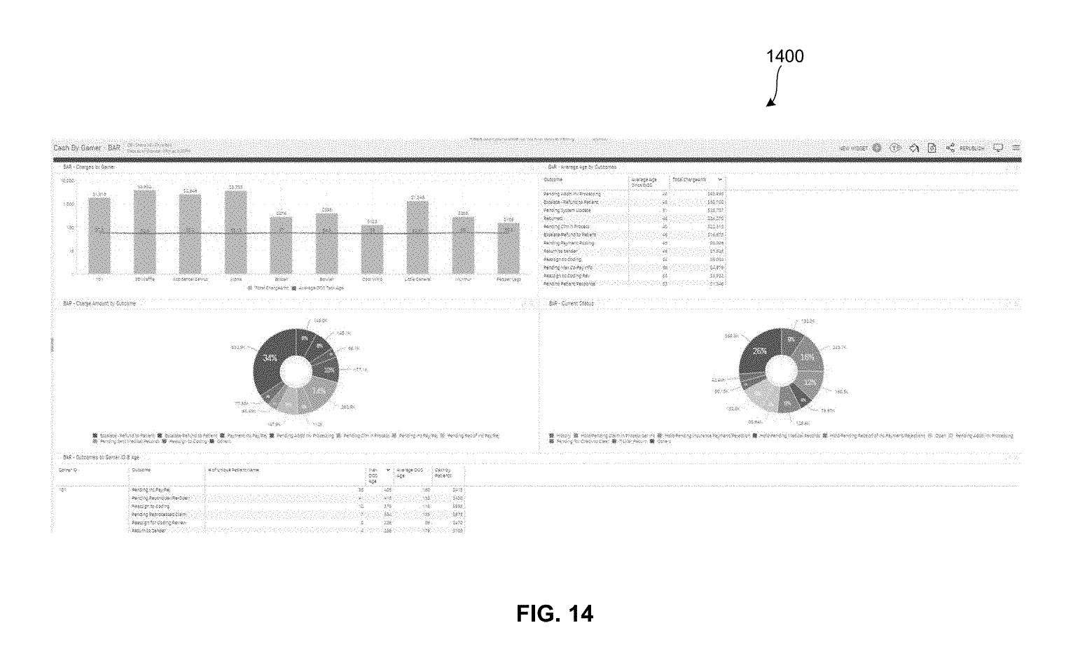

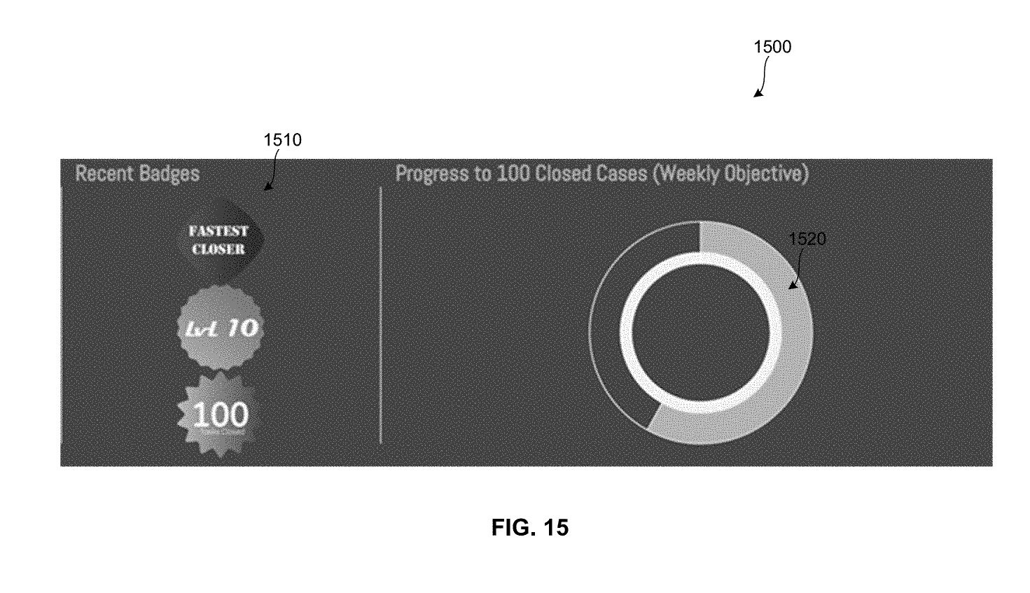

FIG. 15 provides a closer view of an example dashboard 1500 showing recent badges earned 1510 for completion of certain tasks/thresholds, such as fastest closer, top 10, 100 tasks closed, etc. The wheel 1520 in the right-hand portion is a widget representing completion percentage. The example wheel 1520 can be connected to customer user-set goals to represent a current status of completion to give the user a visual representation, for example.

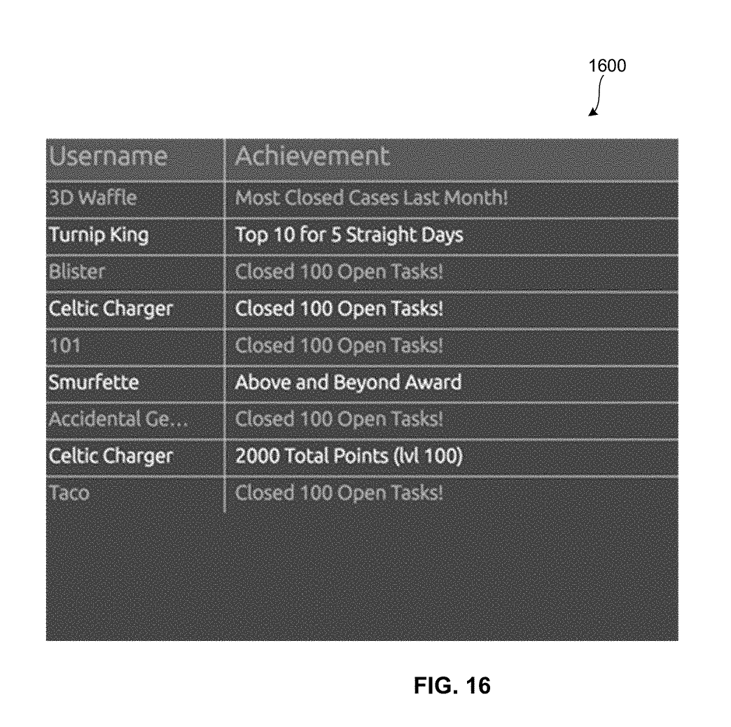

FIG. 16 shows an example achievement feed 1600 for a dashboard. The achievement feed 1600 is a chronological snapshot of the recently earned achievements. The achievement feed 1600 is combines badge tables and compares the task number that triggered the achievement to determine ordering. The example achievement feed 1600 can also be used to filter by self, team, and/or other filter, for example.

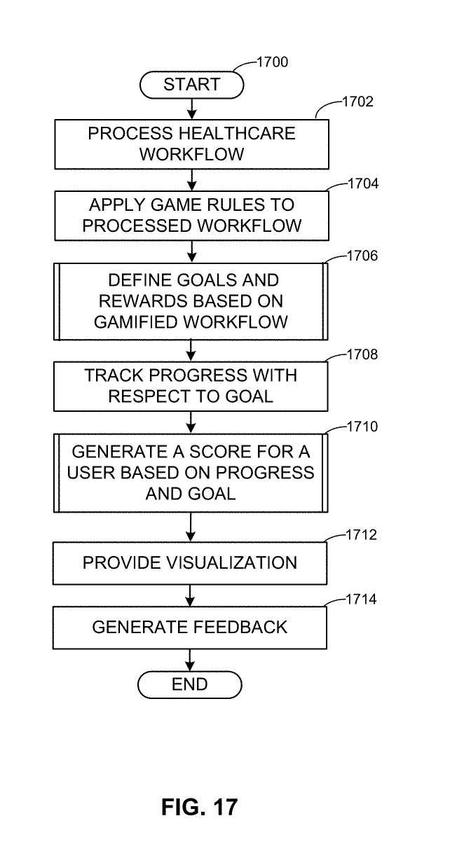

FIG. 17 illustrates a flow diagram of an example method 1700 to generate analytics and provide gamification in a healthcare workflow. At block 1702, a healthcare workflow is processed. For example, a BAR and/or HPA healthcare workflow is processed to divide the workflow into tasks, etc. The tasks are electronically represented within the processed workflow so that tasks can be automatically and electronically analyzed, compared, processed, changed, etc.

At block 1704, game rules are applied to the processed workflow. For example, rules, scoring values, weights, etc., are applied to each task in the workflow such that a user completing that task receives a score (e.g., which may be weighted, etc.). Values, rules, weights, etc., can be electronically represented with the associated task(s) in the processed workflow for automated, electronic processing, comparison, manipulation, etc.

At block 1706, goals and rewards are defined. For example, based on values, weights, etc., assigned to each task, goals and rewards are aligned with workflow task and associated scores, etc. In certain examples, goal(s) and reward(s) can be electronically represented with respect to the gamified workflow for automated electronic processing and comparison of progress to goal(s), etc.

At block 1708, progress is tracked with respect to goal(s). For example, a user and/or group of users is monitored in the organization to track healthcare workflow execution and associated progress toward one of the defined goal(s). User/group progress can be monitored based on executed application(s), entered data, electronic checklist, incoming sensor data, etc.

At block 1710, a score is generated based on the determined progress toward the goal. For example, based on the value, weight, etc., assigned to a task and a measure of whether or not the user has completed the task, is close to completing the task, is a certain percentage along completing the task, is behind in completing the task, etc., a score is assigned. Per task and/or total user scores can be calculated according to Equations 1 and 2 described above, for example. A composite score can be accumulated and/or otherwise generated by the score calculator 730, etc., based on multiple tasks, for example.

At block 1712, a visualization is provided based on the monitored progress, goal, reward, and score. For example, one or more dashboards can be generated automatically and electronically by the system (e.g., the game engine 530 and data cube 540, etc.) to show user progress, organizational status, leaderboard, etc.

At block 1714, feedback can be generated to update goal(s), reward(s), progress, score(s), etc., based on the tracking and analysis of blocks 1702-1710. The feedback can be used to update and/or otherwise modify a healthcare workflow to be monitored (block 1702), game rule(s) (block 1704), goal(s) and/or reward(s) (block 1706), progress (block 1708), score generation (block 1710), visualization (block 1712), etc. Thus, feedback can alter, update, and/or improve operation at one or more blocks in the example workflow 1700 to allow the workflow to dynamically, automatically update and improve. The feedback can be based on a comparison of the score to the goal to provide feedback (e.g., based on meeting the goal, not meeting the goal, almost meeting the goal, easily meeting the goal, etc.). Feedback can also include facilitating the reward to be provided to the user if the goal(s) were met, for example. Thus, the example workflow 1700 provides a technological improvement in system-driven workflow monitoring, user tracking, and incentivization through improvements in tracking, quantization of goals with respect to workflow activities, score generation, visualization of progress and/or outcomes, and associated analytics.

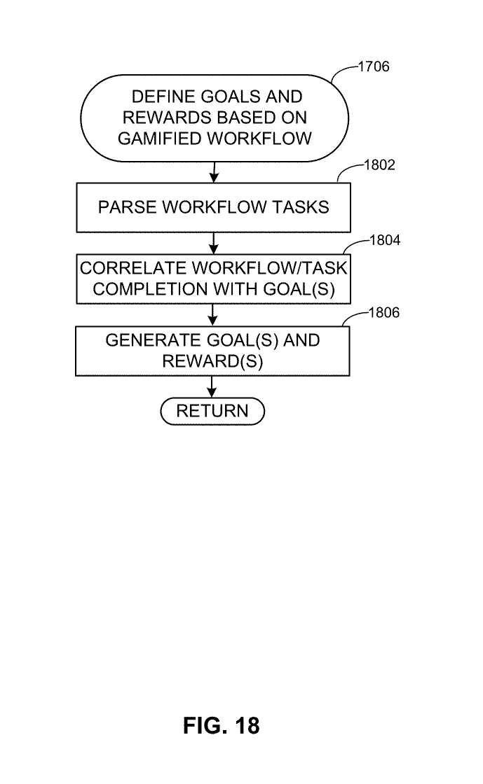

FIG. 18 illustrates a flow diagram of an example implementation of block 1706 of the example process 1700 to define goals and rewards based on a gamified workflow. At block 1802, the workflow tasks are parsed based on the associated game rules, scoring values, weights, etc. For example, each task in a workflow for a user, team, group, and/or organization is parsed based on game rule(s), scoring value(s), weight(s), etc., that were assigned to that task at block 1704.

At block 1804, completion of the workflow(s) and associated task(s) is correlated with organizational, team/group, and/or individual goals (e.g., to see n patients in a given shift, to read×exams, to process labs in y time, to reduce errors in orders by z percent, to have 100% participation in quality review, to reward the practitioner with the fewest readmissions, to reduce billing delays by c %, to achieve 99% reimbursement, etc.).

At block 1806, goals and rewards are generated for an individual user, group/team of users, organization, etc., based on priority of workflow(s) and/or associated task(s) to the organization, team/group, individual, etc. Based on the importance (and associated weight, score, etc.), goal(s) can be generated and associated reward(s) can be assigned based on the value or other importance of that goal. For example, a certain goal may be more important to the organization than another goal, so that first goal is assigned a greater reward than the second goal. As another example, a first goal may be more difficult to achieve than a second goal, so the first goal is assigned a greater reward for completion. In some examples, an optional task in a workflow may be desirable to complete but not required. Additional reward can be given for those who optionally complete that task, for example. Thus, gamification including point scoring, competition with others (and/or set goal(s), threshold(s), and/or other criteria), etc., according to defined rules of play provide incentives and monitoring for improved, automated, dynamic workflow and workforce management, for example.

The goal(s) and reward(s) can then be provided to block 1708 to track progress with respect to those goal(s).

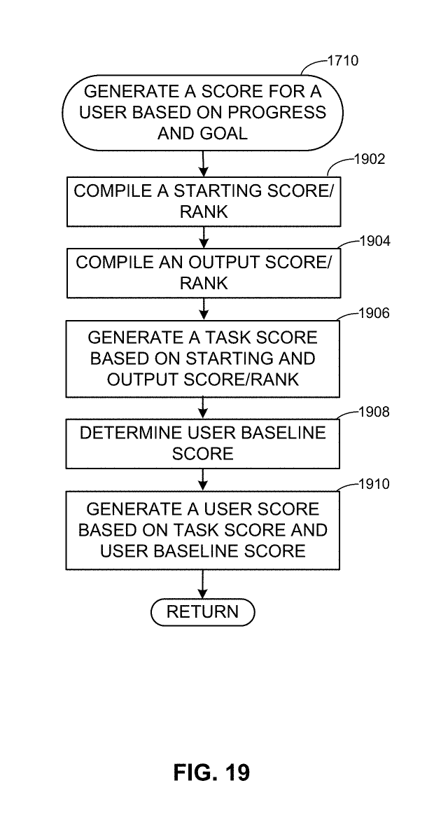

FIG. 19 illustrates a flow diagram of an example implementation of block 1710 of the example process 1700 to generate a score for a user based on progress and goal. At block 1902, a starting score and/or rank is compiled for a task based on organizational scoring data and team scoring data. The starting point score/rank can be for a particular user and/or for any user in the organization and/or team/group, etc. The starting score can be based on goal(s), workflow(s) involving the task, etc.

At block 1904, an output or completion score and/or rank is compiled for the task. For example, the output/completion score/rank can be based on the particular user and/or for any user in the organization and/or team/group, etc. The output/completion score/rank can be determined based on how the task fits into the workflow and/or goal(s) for the organization, group, team, etc., for example.

At block 1906, a task score is generated based on the starting score/rank and the output/completion score/rank. For example, as described in connection with Equation 1 above, the task score can be generated by multiplying the starting point score/rank (e.g., from the organization and/or team/group, etc.) by the output score/rank (e.g., from the organization and/or team/group, etc.).

At block 1908, a baseline score is determined for the user (e.g., based on an initial starting value for that organization, group, team, etc., based on a prior score for that user, etc.). At block 1910, a user score is generated by combining the task score with the baseline score to determine the user's total score (e.g., as described in connection with Equation 2 above). The score is provided to block 1712 for visualization, analytics, feedback, etc.

Thus, certain examples provide a revenue velocity dashboard to improve revenue velocity and provide transparency of data metric(s) that impact the revenue cycle through focus areas such as a decrease in number of accounts receivable days, decrease in charge lag days, decrease in denials and rejections, etc.

Certain examples provide an escalations/reassignments dashboard to improve turnaround to resolve issues (e.g., denials, rejections, refunds, etc.) and reduce or minimize escalations/reassignment of an invoice by reducing a number of touches with tasks and invoices, identifying escalation trends to promote education improvements, provide visibility to evaluate when to empower/adjust employee threshold authorization to gain efficiency, etc.

Certain examples provide a hold bills dashboard to improve evaluation of held invoices (e.g., eligibility, insurance, etc.) and data trends to improve hold bill productivity through visibility to hold bills, status, aging, etc., evaluation of hold bill logic to determine relevance of workflow, analysis of trends of hold bill types and staff productivity, etc.

Certain examples provide a cash collection dashboard to improve analysis of data to proactively identify and monitor patterns to increase cash flow through reducing errors in claims going out, decrease lag days of getting charges from a provider, more efficiently address rejection of denials, more timely filing to payers, availability to obtain payer reimbursements to user for contracting, etc.

Certain examples provide a revenue cycle productivity dashboard to improve productivity standards by work process and ability to redistribute work through insight into data to define and measure productivity incentives, identify and analyze trends and improvement opportunity for staff training and allocation, etc.

Other dashboards that can be provided include management dashboards such as billing and accounts receivable, revenue cycle, hospital patient accounting, etc.

Certain examples help improve operations by helping to improve efficiency and financial performance through gamification evaluations and incentives. Certain example uncover opportunities for improvement through identification of success, failure, challenge, pattern, tendency, etc., identified through gamification and resulting leader boards, dashboards, etc. Certain examples facilitate generation of an action plan to improve poor/unsatisfactory results through incentives, competition, and other gamification mechanisms.

For example, in revenue cycle management, gamification drives productivity, employee satisfaction, cash flow, denial management, accounts receivable days, etc. Operations can be automatically optimized and/or otherwise improved through the technological improvements of the present disclosure to facilitate efficiency and performance improvement, for example.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

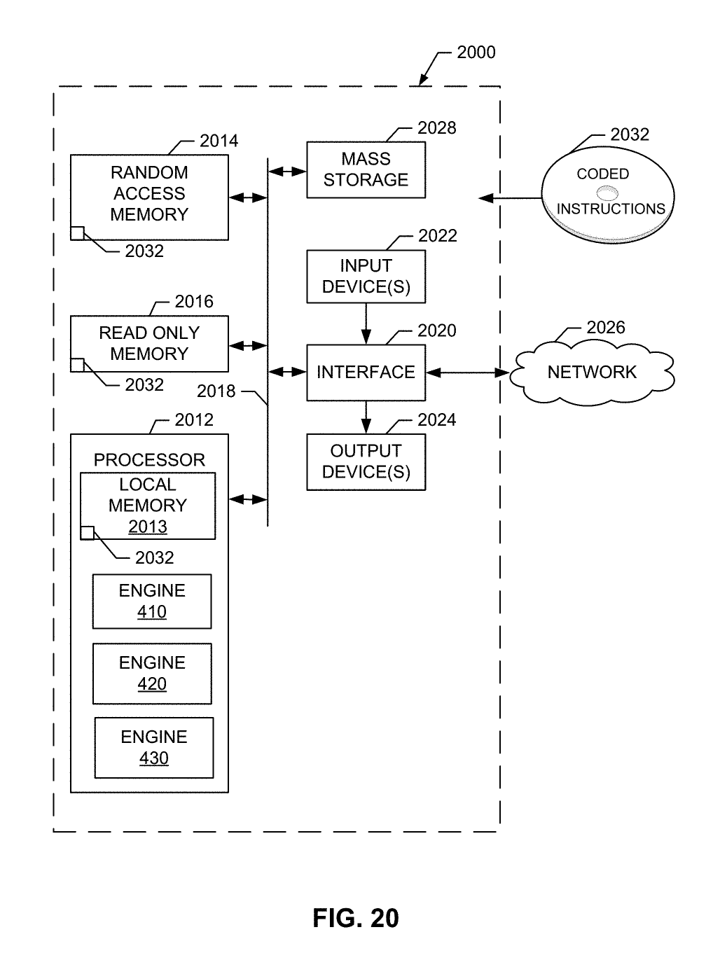

FIG. 20 is a block diagram of an example processor platform 2000 capable of executing instructions to implement the example systems and methods of FIGS. 1-19. The processor platform 2000 can be, for example, a server, a personal computer, a mobile device (e.g., a cell phone, a smart phone, a tablet such as an IPAD™), a personal digital assistant (PDA), an Internet appliance, or any other type of computing device.

The processor platform 2000 of the illustrated example includes a processor 2012. Processor 2012 of the illustrated example is hardware. For example, processor 2012 can be implemented by one or more integrated circuits, logic circuits, microprocessors or controllers from any desired family or manufacturer.

Processor 2012 of the illustrated example includes a local memory 2013 (e.g., a cache). Processor 2012 of the illustrated example is in communication with a main memory including a volatile memory 2014 and a non-volatile memory 2016 via a bus 2018. Volatile memory 2014 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 2016 can be implemented by flash memory and/or any other desired type of memory device. Access to main memory 2014, 2016 is controlled by a memory controller. The processor 2012, alone or in conjunction with the memory 2013, can be used to implement the example organization review and goal setting engine 410, processing tracking engine 420, reward and behavior modification engine 430, and/or other part of the systems disclosed herein.

Processor platform 2000 of the illustrated example also includes an interface circuit 2020. Interface circuit 2020 can be implemented by any type of interface standard, such as an Ethernet interface, a universal serial bus (USB), and/or a PCI express interface.

In the illustrated example, one or more input devices 2022 are connected to the interface circuit 2020. Input device(s) 2022 permit(s) a user to enter data and commands into processor 2012. The input device(s) 2022 can be implemented by, for example, an audio sensor, a microphone, a camera (still or video), a keyboard, a button, a mouse, a touchscreen, a track-pad, a trackball, isopoint and/or a voice recognition system.

One or more output devices 2024 are also connected to interface circuit 2020 of the illustrated example. Output devices 2024 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). Interface circuit 2020 of the illustrated example, thus, typically includes a graphics driver card, a graphics driver chip or a graphics driver processor.

Interface circuit 2020 of the illustrated example also includes a communication device such as a transmitter, a receiver, a transceiver, a modem and/or network interface card to facilitate exchange of data with external machines (e.g., computing devices of any kind) via a network 2026 (e.g., an Ethernet connection, a digital subscriber line (DSL), a telephone line, coaxial cable, a cellular telephone system, etc.).

Processor platform 2000 of the illustrated example also includes one or more mass storage devices 2028 for storing software and/or data. Examples of such mass storage devices 2028 include floppy disk drives, hard drive disks, compact disk drives, Blu-ray disk drives, RAID systems, and digital versatile disk (DVD) drives.

Coded instructions 2032 associated with any of FIGS. 1-19 can be stored in mass storage device 2028, in volatile memory 2014, in the non-volatile memory 2016, and/or on a removable tangible computer readable storage medium such as a CD or DVD.

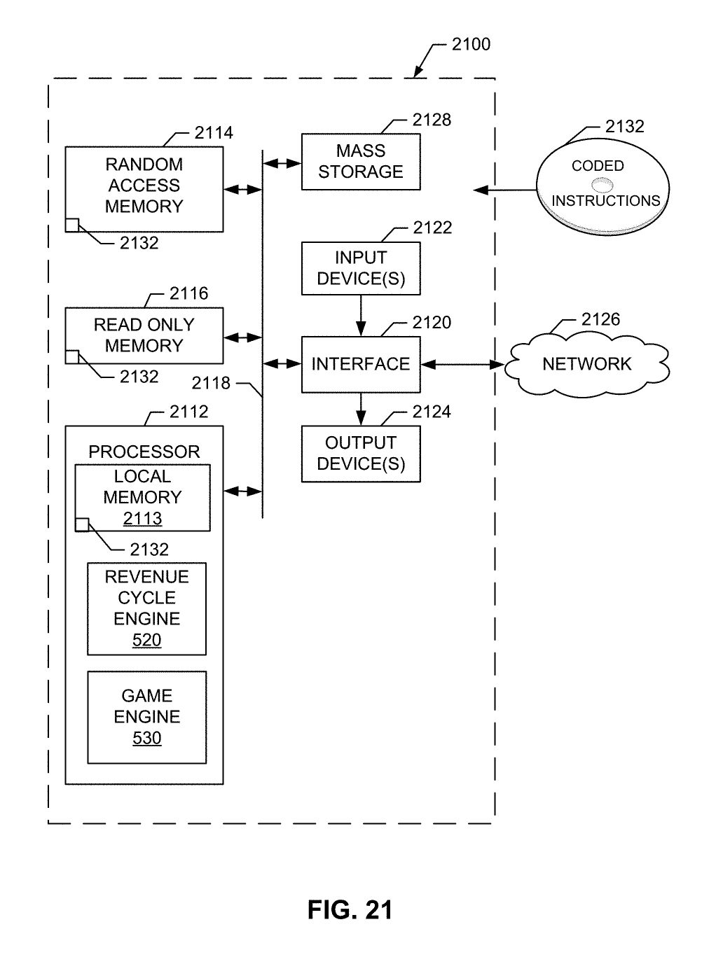

FIG. 21 is a block diagram of an example processor platform 2100 capable of executing instructions to implement the example systems and methods of FIGS. 1-19. The processor platform 2100 can be, for example, a server, a personal computer, a mobile device (e.g., a cell phone, a smart phone, a tablet such as an IPAD′), a personal digital assistant (PDA), an Internet appliance, or any other type of computing device.

The processor platform 2100 of the illustrated example includes a processor 2112. Processor 2112 of the illustrated example is hardware. For example, processor 2112 can be implemented by one or more integrated circuits, logic circuits, microprocessors or controllers from any desired family or manufacturer.

Processor 2112 of the illustrated example includes a local memory 2113 (e.g., a cache). Processor 2112 of the illustrated example is in communication with a main memory including a volatile memory 2114 and a non-volatile memory 2116 via a bus 2118. Volatile memory 2114 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 2116 can be implemented by flash memory and/or any other desired type of memory device. Access to main memory 2114, 2116 is controlled by a memory controller. The processor 2112, alone or in conjunction with the memory 2113, can be used to implement the revenue cycle management engine 520, the game engine 530 and/or other part of the systems disclosed herein.

Processor platform 2100 of the illustrated example also includes an interface circuit 2120. Interface circuit 2120 can be implemented by any type of interface standard, such as an Ethernet interface, a universal serial bus (USB), and/or a PCI express interface.

In the illustrated example, one or more input devices 2122 are connected to the interface circuit 2120. Input device(s) 2122 permit(s) a user to enter data and commands into processor 2112. The input device(s) 2122 can be implemented by, for example, an audio sensor, a microphone, a camera (still or video), a keyboard, a button, a mouse, a touchscreen, a track-pad, a trackball, isopoint and/or a voice recognition system.

One or more output devices 2124 are also connected to interface circuit 2120 of the illustrated example. Output devices 2124 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). Interface circuit 2120 of the illustrated example, thus, typically includes a graphics driver card, a graphics driver chip or a graphics driver processor.

Interface circuit 2120 of the illustrated example also includes a communication device such as a transmitter, a receiver, a transceiver, a modem and/or network interface card to facilitate exchange of data with external machines (e.g., computing devices of any kind) via a network 2126 (e.g., an Ethernet connection, a digital subscriber line (DSL), a telephone line, coaxial cable, a cellular telephone system, etc.).

Processor platform 2100 of the illustrated example also includes one or more mass storage devices 2128 for storing software and/or data. Examples of such mass storage devices 2128 include floppy disk drives, hard drive disks, compact disk drives, Blu-ray disk drives, RAID systems, and digital versatile disk (DVD) drives.

Coded instructions 2132 associated with any of FIGS. 1-19 can be stored in mass storage device 2128, in volatile memory 2114, in the non-volatile memory 2116, and/or on a removable tangible computer readable storage medium such as a CD or DVD.

It may be noted that operations performed by the processor platform 2000 (e.g., operations corresponding to process flows or methods discussed herein, or aspects thereof) may be sufficiently complex that the operations may not be performed by a human being within a reasonable time period.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. An apparatus comprising:
a game engine including a processor configured to at least:
process a healthcare workflow to form a processed workflow organized according to task, each task electronically represented in the processed workflow;
apply game rules to the processed workflow to form a gamified workflow, the game rules associating a value with each task in the processed workflow, the value electronically represented with the corresponding task in the gamified workflow; and
define a goal and a reward based on the gamified workflow; and
a process tracker including a processor configured to at least:
track progress with respect to the goal during execution of the gamified workflow including each task;
generate a score based on the monitored progress of the execution of the gamified workflow; and
provide feedback and reward based on a comparison of the score to the goal.

2. The apparatus of claim 1, further including a data cube to organize and store information from the game engine for tracking, visualization, and analytics.

3. The apparatus of claim 2, wherein the game engine and data cube provide a visualization including at least one of a user board, a leader board, or an analytics dashboard.

4. The apparatus of claim 1, further including a reward and behavior modification engine to provide feedback based on the score, goal, and game rules.

5. The apparatus of claim 1, wherein the value, goal, and reward are based on at least one of an organizational goal, a team goal, or an individual goal.

6. The apparatus of claim 1, further including a score calculator to calculate a per task calculation based on a starting a score and an output score and to calculate a user score based on a baseline score and the task score.

7. The apparatus of claim 1, wherein the game engine includes a health data extractor to extract health-related data for the healthcare workflow, a data processor to process data associated with the healthcare workflow, a game rule applicator to apply game rules to the tasks associated with the healthcare workflow, and a data insight formatter to extract feedback from the gamified workflow, associated tasks, score, and reward.

8. A method comprising:
processing, using a game engine including a processor, a healthcare workflow to form a processed workflow organized according to task, each task electronically represented in the processed workflow;
applying, using the processor, game rules to the processed workflow to form a gamified workflow, the game rules associating a value with each task in the processed workflow, the value electronically represented with the corresponding task in the gamified workflow;
defining, using the processor, a goal and a reward based on the gamified workflow;
tracking, using the processor, progress with respect to the goal during execution of the gamified workflow including each task;
generating, using the processor, a score based on the monitored progress of the execution of the gamified workflow; and
providing, using the processor, feedback and reward based on a comparison of the score to the goal.

9. The method of claim 8, further including providing a visualization including at least one of a user board, a leader board, or an analytics dashboard.

10. The method of claim 8, wherein the value, goal, and reward are based on at least one of an organizational goal, a team goal, or an individual goal.

11. The method of claim 8, wherein generating a score further includes:
calculating a per task calculation based on a starting score and an output score; and
calculating a user score based on a baseline score and the task score.

12. The method of claim 11, wherein the baseline score is at least one of a user baseline score, a team baseline score, or an organizational baseline score.

13. The method of claim 11, wherein the starting score and the output score are provided by at least one of an organizational scoring system or a team scoring system.

14. A computer-readable storage medium including instructions which, when executed, configure a processor to at least implement a system including:
a game engine configured to at least:
process a healthcare workflow to form a processed workflow organized according to task, each task electronically represented in the processed workflow;
apply game rules to the processed workflow to form a gamified workflow, the game rules associating a value with each task in the processed workflow, the value electronically represented with the corresponding task in the gamified workflow; and
define a goal and a reward based on the gamified workflow; and
a process tracker configured to at least:
track progress with respect to the goal during execution of the gamified workflow including each task;
generate a score based on the monitored progress of the execution of the gamified workflow; and
provide feedback and reward based on a comparison of the score to the goal.

15. The computer-readable storage medium of claim 14, wherein the instructions, when executed, further configure the processor to implement a data cube to organize and store information from the game engine for tracking, visualization, and analytics.

16. The computer-readable storage medium of claim 15, wherein the game engine and data cube provide a visualization including at least one of a user board, a leader board, or an analytics dashboard.

17. The computer-readable storage medium of claim 14, wherein the instructions, when executed, further configure the processor to implement a reward and behavior modification engine to provide feedback based on the score, goal, and game rules.

18. The computer-readable storage medium of claim 14, wherein the value, goal, and reward are based on at least one of an organizational goal, a team goal, or an individual goal.

19. The computer-readable storage medium of claim 14, wherein the instructions, when executed, further configured the processor to implement a score calculator to calculate a per task calculation based on a starting a score and an output score and to calculate a user score based on a baseline score and the task score.

20. The computer-readable storage medium of claim 14, wherein the game engine includes a health data extractor to extract health-related data for the healthcare workflow, a data processor to process data associated with the healthcare workflow, a game rule applicator to apply game rules to the tasks associated with the healthcare workflow, and a data insight formatter to extract feedback from the gamified workflow, associated tasks, score, and reward.
